(19) United States
(12) Patent Application Publication
(10) Pub. No.: US 2026/0061126 A1
(43) Pub. Date: Mar. 5, 2026

(54) HYPERTENSION PREDICTION

(71) Applicant: Becton, Dickinson and Company, Franklin Lakes, NJ (US)

(72) Inventors: Sai Prasad Buddi, Santa Ana, CA (US); Xianfu Liu, Irvine, CA (US)

(21) Appl. No.: 19/312,152

(22) Filed: Aug. 27, 2025

Related U.S. Application Data
(60) Provisional application No. 63/687,719, filed on Aug. 27, 2024.

(51) Int. Cl. A61M 5/172; G16H 50/20

(52) U.S. Cl. CPC A61M 5/1723; G16H 50/20; A61M 2205/502; A61M 2230/30

(57) ABSTRACT

A system for predicting acute hypertension for a patient includes a hemodynamic sensor that produces, on an ongoing basis, a hemodynamic sensor signal representative of an arterial pressure waveform of the patient and an integrated hardware unit. The integrated hardware unit includes a system processor, a system memory, and a display including a user interface. The system memory includes instructions that, when executed by the system processor, cause the system to receive the hemodynamic sensor signal representative of the arterial pressure waveform of the patient; extract features from the arterial pressure waveform of the patient; determine, by a machine learning model, a probability of an acute hypertensive event of the patient based on the features extracted from the arterial pressure waveform; and output, to the display, an indication of the probability of the acute hypertensive event of the patient.

### CROSS-REFERENCE TO RELATED APPLICATION(S)

This application claims the benefit of U.S. Provisional Application No. 63/687,719, filed Aug. 27, 2024, and entitled “HYPERTENSION PREDICTION,” the disclosure of which is hereby incorporated by reference in its entirety.

### BACKGROUND

The present disclosure relates generally to hemodynamic monitoring and, more specifically, to predicting hypertension in a patient using monitored hemodynamic data.

Hypertension, or high blood pressure, is defined as elevated blood pressure above a certain level. Hypertension in a critical care setting (i.e., acute hypertension), such as during surgery or an intensive care unit (ICU) stay, can be physiologically different than the lifestyle disease. That is, a person may typically have normal blood pressure but may experience fluctuations in those settings.

Acute hypertension is of concern to clinicians because an acute hypertensive event may indicate that the patient is experiencing pain (e.g., due to inadequate anesthesia) or has an underlying heart condition, among other reasons. More generally, blood pressure outside the autoregulation range of blood pressure can result in cell damage. Additionally, acute hypertension during surgery can cause excessive bleeding. Studies have associated acute hypertension with negative surgical outcomes, such as length of hospital stay, postoperative delirium, and stroke, among others.

### SUMMARY

In one example, a system for predicting acute hypertension for a patient includes a hemodynamic sensor that produces, on an ongoing basis, a hemodynamic sensor signal representative of an arterial pressure waveform of the patient and an integrated hardware unit. The integrated hardware unit includes a system processor, a system memory, and a display including a user interface. The system memory includes instructions that, when executed by the system processor, cause the system to receive the hemodynamic sensor signal representative of the arterial pressure waveform of the patient; extract features from the arterial pressure waveform of the patient; determine, by a machine learning model, a probability of an acute hypertensive event of the patient based on the features extracted from the arterial pressure waveform; and output, to the display, an indication of the probability of the acute hypertensive event of the patient.

In another example, a method for predicting acute hypertension for a patient in a system including an integrated hardware unit that includes a system processor, a system memory, and a display including a user interface includes receiving, on an ongoing basis from a hemodynamic sensor, a hemodynamic sensor signal representative of an arterial pressure waveform of the patient. The method further includes extracting features from the arterial pressure waveform of the patient and determining, by a machine learning model, a probability of an acute hypertensive event of the patient based on the features extracted from the arterial pressure waveform. The method further includes outputting, to the display, an indication of the probability of the acute hypertensive event of the patient.

### BRIEF DESCRIPTION OF THE DRAWINGS

FIG. 1 is a schematic block diagram illustrating an example hemodynamic monitoring system that determines a likelihood that a patient will experience an acute hypertensive event based on hemodynamic data from the patient.

FIG. 2 is a perspective view of an example hemodynamic monitor that includes a hardware processor that determines a likelihood that a patient will experience an acute hypertensive event.

FIG. 3 is a perspective view of an example minimally invasive pressure sensor that can be attached to a patient for sensing hemodynamic data representative of arterial pressure of the patient.

FIG. 4 is a perspective view of an example non-invasive sensor for sensing hemodynamic data representative of arterial pressure of a patient.

FIG. 5 is a schematic block diagram illustrating modules of the hemodynamic monitoring system.

FIG. 6 is a graph illustrating an example trace of an arterial pressure waveform including example indicia corresponding to a likelihood that a patient will experience an acute hypertensive event.

FIG. 7 is a schematic block diagram illustrating a logistic model of the hypertension prediction algorithm.

FIG. 8 is a schematic block diagram illustrating a TTE DL model of the hypertension prediction algorithm.

FIG. 9 is a graph illustrating mean arterial pressure (MAP) over time and showing a time to event (TTE).

FIG. 10 is a schematic block diagram illustrating an example architecture of a deep learning model that may be used by the TTE DL model.

FIG. 11A is a graph illustrating a first trace of MAP over time.

FIG. 11B is a graph illustrating probabilities over time predicted by the logistic model, the TTE DL model, and a combined model, and corresponding to the first trace of MAP over time.

FIG. 12 is a schematic block diagram illustrating an example training system for training a hypertension prediction algorithm.

FIG. 13 is a schematic block diagram illustrating another example training system for training a hypertension prediction algorithm.

FIG. 14 is a schematic block diagram illustrating an example training system and computer-readable medium including instructions for performing model training.

### DETAILED DESCRIPTION

According to techniques of this disclosure, systems and methods for predicting acute hypertensive events in patients in a critical care setting use mean arterial pressure (MAP) readings with a logistic regression model and/or a deep learning model to determine whether a future hypertensive event will occur and/or whether it will occur within a certain time period.

Acute hypertension can be defined in numerous ways, e.g., based on literature and other recommendations. Generally, “normal” blood pressure is 120 mmHg systolic/80 mmHg diastolic pressure, although other guidelines may also be used, depending on the setting. During surgery (i.e., intra-operative), some literature sources use MAP to define hypertension (e.g., MAP>100 mmHg or MAP>105 mmHg). MAP is an average calculated arterial blood pressure for a single cardiac cycle. Various methods may be used to estimate MAP. A common calculation estimates MAP according to the following formula:

$$MAP = DP + 1 / 3 ( PP ) \qquad ( Equation 1 )$$

Where,
DP is diastolic pressure; and
PP is pulse pressure, which is equal to the difference between systolic pressure and diastolic pressure.
There are also other literature recommendations for specific settings, e.g., during cardiac surgery patients are recommended to be kept between 70-100 mmHg MAP, and during carotid endarterectomy surgery patients are recommended to be kept within 120% of a MAP baseline. MAP has also been used to define hypotension (e.g., MAP<65 mmHg).

Thus, MAP can be used to define acute hypertension. MAP may be useful for defining acute hypertension because MAP reflects perfusion pressure and the limits of autoregulation. Moreover, some studies have suggested that MAP may be a better predictor of hypertension-related vascular alteration compared to systolic/diastolic pressure. A high MAP also generally reflects a high systolic and/or diastolic pressure (120 mmHg systolic/80 mmHg diastolic ≈95 mmHg MAP and 160 mmHg systolic/90 mmHg diastolic ≈115 mmHg MAP). For example, 115 mmHg can be selected as a threshold, where MAP≥115 mmHg represents an acute hypertensive event. Other thresholds, such as thresholds based on a percent change in MAP and/or variable thresholds, are also possible for patient-specific blood pressure management.

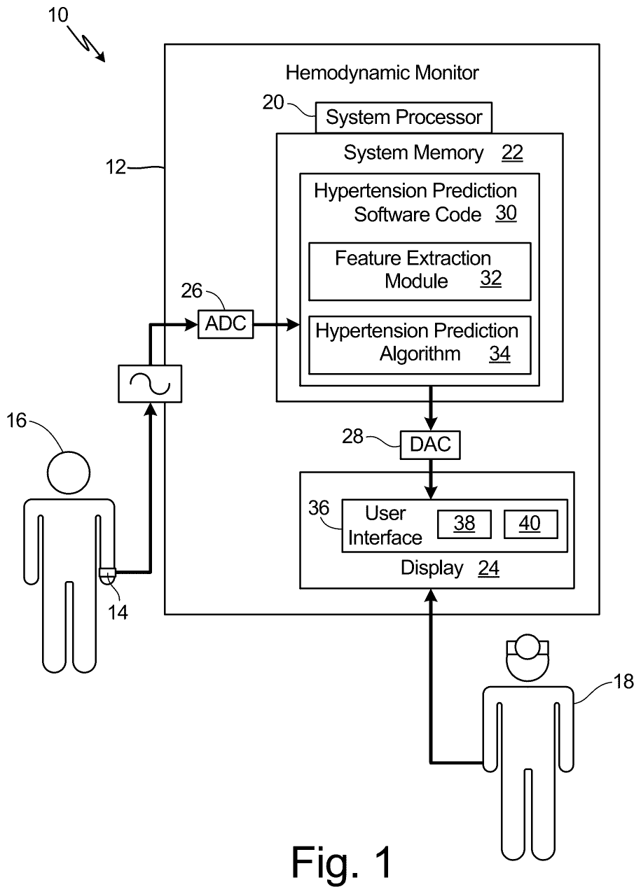

FIG. 1 is a schematic block diagram of hemodynamic monitoring system 10 that determines a likelihood that a patient will experience an acute hypertensive event based on hemodynamic data from the patient. FIG. 1 shows hemodynamic monitoring system 10, including hemodynamic monitor 12 and hemodynamic sensor 14. Hemodynamic monitor 12 includes system processor 20, system memory 22, display 24, analog-to-digital converter (ADC) 26, and digital-to-analog converter (DAC) 28. System memory 22 includes hypertension prediction software code 30, which includes feature extraction module 32 and hypertension prediction algorithm 34. Display 24 includes user interface 36, which includes control elements 38 and sensory alarm 40. FIG. 1 also shows patient 16 and healthcare worker 18.

As illustrated in FIG. 1, hemodynamic monitoring system 10 includes hemodynamic monitor 12 and hemodynamic sensor 14. Hemodynamic monitoring system 10 can be implemented within a patient care environment, such as an ICU, an OR, or other patient care environment, for monitoring a hemodynamic condition of a patient. As illustrated in FIG. 1, the patient care environment can include patient 16 and healthcare worker 18 trained to utilize hemodynamic monitoring system 10.

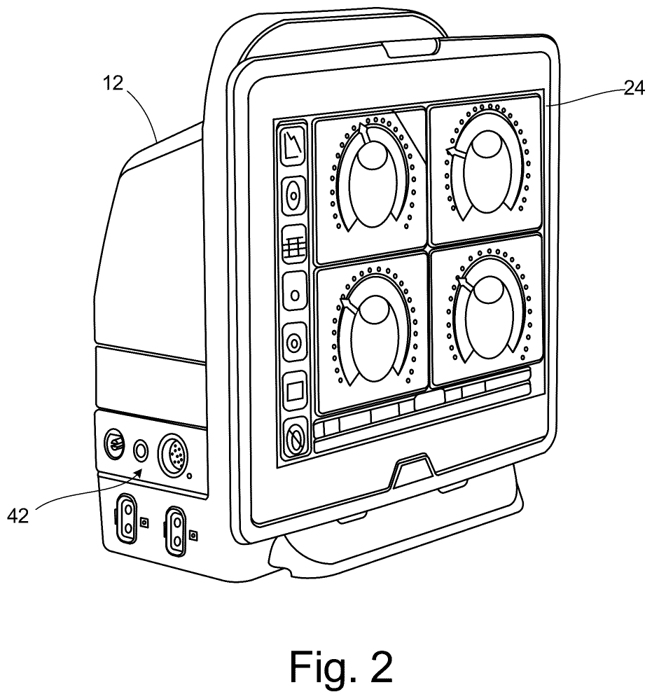

Hemodynamic monitor 12, as described below with respect to FIG. 2, can be an integrated hardware unit including system processor 20, system memory 22, display 24, ADC 26, and DAC 28 all contained within a housing. In other examples, any one or more components and/or described functionality of hemodynamic monitor 12 can be distributed among multiple hardware units. For instance, in some examples, display 24 can be a separate display device that is remote from and operatively coupled with hemodynamic monitor 12. In general, although illustrated and described in the example of FIG. 1 as an integrated hardware unit, it should be understood that hemodynamic monitor 12 can include any combination of devices and components that are electrically, communicatively, or otherwise operatively connected to perform functionality attributed herein to hemodynamic monitor 12.

As illustrated in FIG. 1, system memory 22 stores hypertension prediction software code 30. Hypertension prediction software code 30 includes feature extraction module 32 and hypertension prediction algorithm 34. Display 24 provides user interface 36, which includes control elements 38 that enable user interaction with hemodynamic monitor 12 and/or other components of hemodynamic monitoring system 10. User interface 36, as illustrated in FIG. 1, also provides sensory alarm 40 to provide warning to medical personnel if hemodynamic monitoring system 10 determines a hypertension index that is indicative of a likelihood that patient 16 will experience an acute hypertensive event, as is further described below.

Hemodynamic sensor 14 can be attached to patient 16 to sense hemodynamic data representative of an arterial pressure waveform of patient 16. Hemodynamic sensor 14 is operatively connected to hemodynamic monitor 12 (e.g., electrically and/or communicatively connected via wired or wireless connection, or both) to provide the sensed hemodynamic data to hemodynamic monitor 12. In some examples, hemodynamic sensor 14 provides the hemodynamic data of patient 16 to hemodynamic monitor 12 as an analog signal, which is converted by ADC 26 to digital hemodynamic data representative of an arterial pressure waveform. In other examples, hemodynamic sensor 14 can provide the sensed hemodynamic data to hemodynamic monitor 12 in digital form, in which case hemodynamic monitor 12 may not include or utilize ADC 26. In yet other examples, hemodynamic sensor 14 can provide the hemodynamic data of patient 16 to hemodynamic monitor 12 as an analog signal, which is analyzed in its analog form by hemodynamic monitor 12.

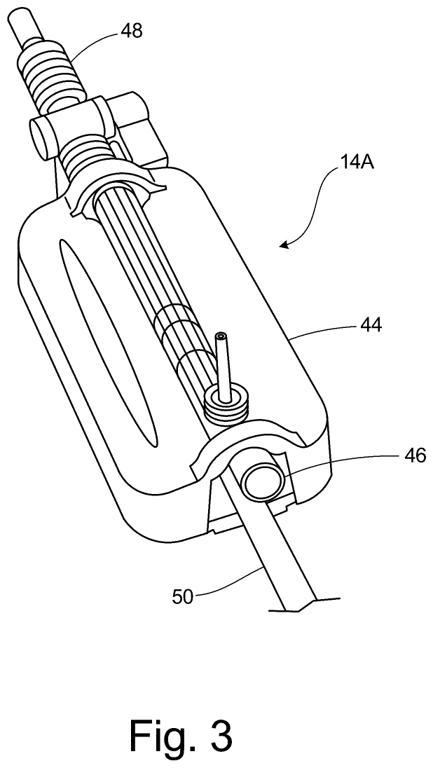

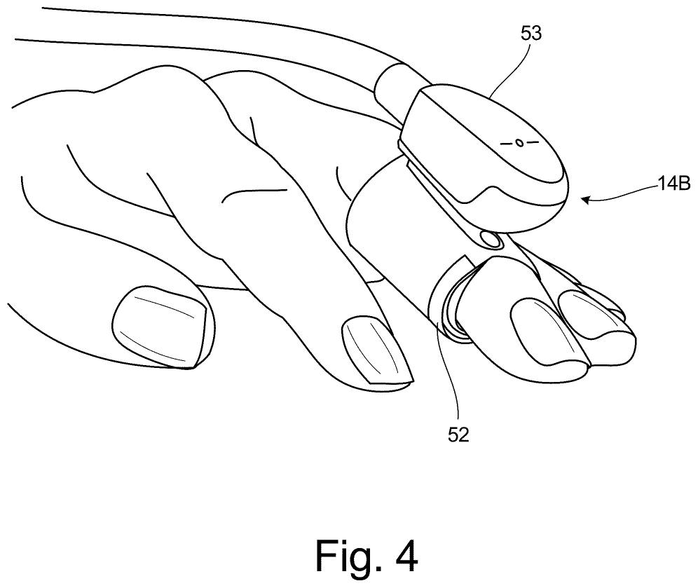

Hemodynamic sensor 14 can include a non-invasive or minimally invasive sensor attached to patient 16. For instance, hemodynamic sensor 14 can take the form of a minimally invasive hemodynamic sensor (e.g., hemodynamic sensor 14A shown in FIG. 3), a non-invasive hemodynamic sensor (e.g., hemodynamic sensor 14B shown in FIG. 4), or another minimally invasive or non-invasive hemodynamic sensor. In some examples, hemodynamic sensor 14 can be attached non-invasively at an extremity of patient 16, such as a forehead, a wrist, an arm, a finger, an ankle, a toe, or other extremity of patient 16. As such, hemodynamic sensor 14 can take the form of a small, lightweight, and comfortable hemodynamic sensor suitable for extended wear by patient 16 to provide substantially continuous beat-to-beat monitoring of the arterial pressure of patient 16 over an extended period of time, such as minutes or hours. In certain examples, hemodynamic sensor 14 can be configured to sense an arterial pressure of patient 16 in a minimally invasive manner. For instance, hemodynamic sensor 14 can be attached to patient 16 via a radial arterial catheter inserted into an arm of patient 16. In other examples, hemodynamic sensor 14 can be attached to patient 16 via a femoral arterial catheter inserted into a leg of patient 16. Such minimally invasive techniques can similarly enable hemodynamic sensor 14 to provide substantially continuous beat-to-beat monitoring of the arterial pressure of patient 16 over an extended period of time, such as minutes or hours.

System processor 20 is configured to execute hypertension prediction software code 30, which implements feature extraction module 32 and hypertension prediction algorithm 34 to produce a hypertension index representing a likelihood that patient 16 will experience an acute hypertensive event. Examples of system processor 20 can include any one or more of a microprocessor, a controller, a digital signal processor (DSP), an application specific integrated circuit (ASIC), a field-programmable gate array (FPGA), or other equivalent discrete or integrated logic circuitry.

System memory 22 can be configured to store information within hemodynamic monitor 12 during operation. System memory 22, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 22 can include volatile and non-volatile computer-readable memories. Examples of volatile memories can include random access memories (RAM), dynamic random-access memories (DRAM), static random-access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

Display 24 can be a liquid crystal display (LCD), a light-emitting diode (LED) display, an organic light-emitting diode (OLED) display, or other display device suitable for providing information to users in graphical form. User interface 36 can include graphical and/or physical control elements that enable user input to interact with hemodynamic monitor 12 and/or other components of hemodynamic monitoring system 10. In some examples, user interface 36 can take the form of a graphical user interface (GUI) that presents graphical control elements presented at, e.g., a touch-sensitive and/or presence sensitive display screen of display 24. In such examples, user input can be received in the form of gesture input, such as touch gestures, scroll gestures, zoom gestures, or other gesture input. In certain examples, user interface 36 can take the form of and/or include physical control elements, such as a physical buttons, keys, knobs, or other physical control elements configured to receive user input to interact with components of hemodynamic monitoring system 10.

In operation, hemodynamic sensor 14 senses hemodynamic data representative of an arterial pressure waveform of patient 16. Hemodynamic sensor 14 provides the hemodynamic data (e.g., as analog sensor data), to hemodynamic monitor 12. ADC 26 converts the analog hemodynamic data to digital hemodynamic data representative of the arterial pressure waveform of the patient.

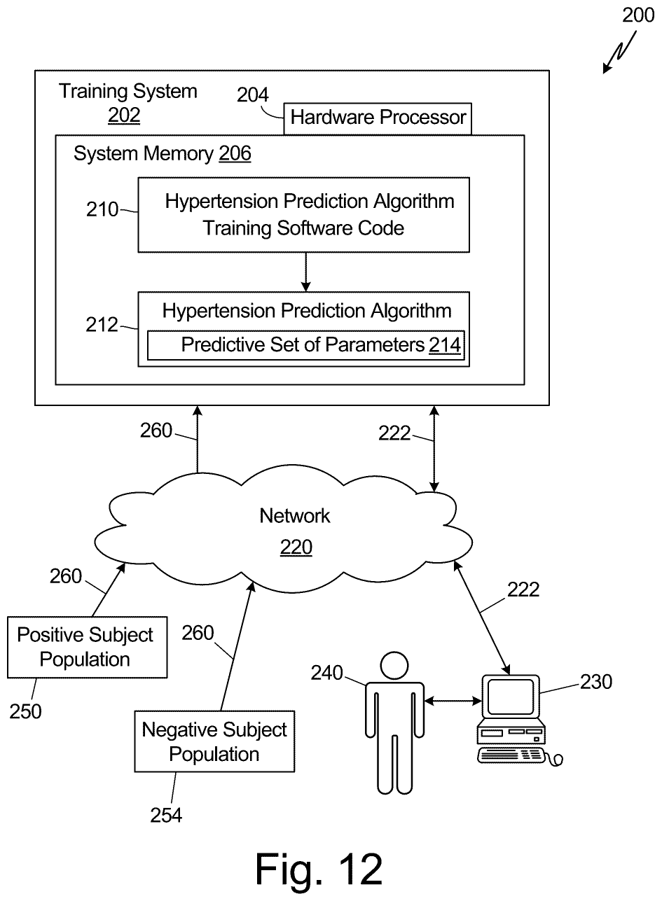

System processor 20 executes hypertension prediction software code 30 to determine, using the received hemodynamic data, a likelihood that patient 16 will experience an acute hypertensive event. For instance, system processor 20 can execute hypertension prediction software code 30 to extract features from the hemodynamic data representative of the blood pressure waveform via feature extraction module 32. One or more blood pressure waveform features can be indicative (or predictive) of the likelihood of a future hypertensive event. Additionally, system processor 20 can execute hypertension prediction software code 30 to determine, via hypertension prediction algorithm 34, a hypertension index representing the likelihood that patient 16 will experience an acute hypertensive event, e.g., within a certain time period. Hypertension prediction algorithm 34 uses the extracted features to determine the hypertension index. Hypertension prediction algorithm 34 includes one or more machine learning models. Features and/or model coefficients utilized by hypertension prediction algorithm 34 can be selected via training operations (as described in greater detail below with reference to FIGS. 12-14) to minimize the error of the hypertension index as predictive of an acute hypertensive event. For example, hypertension prediction software code 30 can determine the hypertension index when a patient is admitted to an ICU, an ER, an OR, etc. and can continuously update the hypertension index during the patient's stay. Alternatively, hypertension prediction software code 30 can determine the hypertension index at discrete times.

System processor 20 executes hypertension prediction software code 30 and can invoke sensory alarm 40 via user interface 36 in response to determining that the hypertension index satisfies predetermined criteria. For instance, hypertension prediction software code 30 can invoke sensory alarm 40 to warn of an acute hypertensive event predicted to occur, such as within the next ten minutes. The hypertension index can be a normalized value between 0 and 100 (or between 0 and 1, or other normalized ranges) with, in some examples, a higher value representing a higher likelihood that patient 16 will experience an acute hypertensive event and a lower value representing a lower likelihood that patient 16 will experience an acute hypertensive event. Sensory alarm 40 can be configured to be invoked if, for example, the hypertension index is greater than 50 (when measured on a normalized scale of 0 to 100) or 0.5 (when measured on a normalized scale of 0 to 1) (i.e., when the prediction is more likely than not). In another example, sensory alarm 40 can be configured to be invoked if the hypertension index is greater than 80 (when measured on a normalized scale of 0 to 100) or 0.8 (when measured on a normalized scale of 0 to 1). In other examples, sensory alarm 40 can be configured to be invoked if the hypertension index is greater than a different threshold. In yet other examples, sensory alarm 40 can be configured to be invoked based on multiple hypertension index thresholds (e.g., different alarms for different thresholds). Sensory alarm 40 can be implemented as one or more of a visual alarm, an audible alarm, a haptic alarm, or other type of sensory alarm. For instance, sensory alarm 40 can be invoked as any combination of flashing and/or colored graphics shown by user interface 36 on display 24, display of the hypertension index via user interface 36 on display 24, a warning sound such as a siren or repeated tone, and a haptic alarm configured to cause hemodynamic monitor 12 to vibrate or otherwise deliver a physical impulse perceptible to healthcare worker 18 or other user.

Accordingly, hemodynamic monitor 12 of hemodynamic monitoring system 10 provides a warning to medical personnel of a likelihood that patient 16 will experience an acute hypertensive event, thereby enabling timely and effective intervention to mitigate or prevent the predicted acute hypertensive event. Techniques described herein therefore increase the usability of hemodynamic monitor 12 by enabling hemodynamic monitor 12 to determine the likelihood that patient 16 will experience an acute hypertensive event, which may be otherwise difficult to accurately predict.

An example of hemodynamic monitor 12 is shown in FIG. 2. An example of a minimally invasive pressure sensor is shown in FIG. 3. An example of a non-invasive pressure sensor is shown in FIG. 4.

FIG. 2 is a perspective view of hemodynamic monitor 12 that determines a likelihood that a patient will experience an acute hypertensive event. As illustrated in FIG. 2, hemodynamic monitor 12 includes display 24 that, in the example shown in FIG. 1, presents a graphical user interface 36 including control elements 38 (e.g., graphical control elements) that enable user interaction with hemodynamic monitor 12. Hemodynamic monitor 12 can also include a plurality of input and/or output (I/O) connectors configured for wired connection (e.g., electrical and/or communicative connection) with one or more peripheral components, such as one or more hemodynamic sensors 14. For instance, as illustrated in FIG. 2, hemodynamic monitor 12 can include I/O connectors 42. Although the example of FIG. 2 illustrates five separate I/O connectors 42, it should be understood that in other examples, hemodynamic monitor 12 can include fewer than five I/O connectors 42 or greater than five I/O connectors 42. In yet other examples, hemodynamic monitor 12 may not include I/O connectors 42, but rather may communicate wirelessly with various peripheral devices.

As described with respect to FIG. 1, hemodynamic monitor 12 includes one or more system processors 20 and computer-readable system memory 22 that stores hypertension prediction software code 30, which is executable to produce a hypertension index representing a likelihood that a patient will experience a future hypertensive event. Hemodynamic monitor 12 can receive sensed hemodynamic data representative of an arterial pressure waveform of the patient, such as via hemodynamic sensor 14 connected to hemodynamic monitor 12 via I/O connectors 42. Hemodynamic monitor 12 executes hypertension prediction software code 30 to determine a hypertension index using the received hemodynamic data and extracted waveform features from the hemodynamic data.

As illustrated in FIG. 1, hemodynamic monitor 12 can present a graphical user interface at display 24. Display 24 can also display an indication of the hypertension index. Display 24 can be an LCD, an LED display, an OLED display, or other display device suitable for providing information to users in graphical form. In some examples, such as the example of FIG. 2, display 24 can be a touch-sensitive and/or presence-sensitive display device configured to receive user input in the form of gestures, such as touch gestures, scroll gestures, zoom gestures, swipe gestures, or other gesture input. Hemodynamic monitor 12 presents control elements that enable user input by, for example, medical personnel.

Hemodynamic monitor 12 receives hemodynamic data from patient 16 via hemodynamic sensor 14. In response to receiving hemodynamic data of patient 16, hemodynamic monitor 12 executes hypertension prediction software code 30 to determine the hypertension index representing a likelihood that a patient will experience an acute hypertensive event using the waveform features and/or model coefficients that were determined during training of hypertension prediction algorithm 34 and display the hypertension index or other indicators on display 24. Additionally, hemodynamic monitor 12 can invoke a sensory alarm, such as an audible alarm, a haptic alarm, or other sensory alarm (e.g., sensory alarm 40 shown in FIG. 1) in response to determining that the hypertension index satisfies predetermined criteria. Accordingly, hemodynamic monitor 12 can provide a warning to alert medical personnel of a predicted future acute hypertensive event of patient 16 prior to patient 16 entering an acute hypertensive state.

FIG. 3 is a perspective view of hemodynamic sensor 14A that can be attached to patient 16 for sensing hemodynamic data representative of arterial pressure of patient 16. Hemodynamic sensor 14A is an example of hemodynamic sensor 14 (shown in FIG. 1). Hemodynamic sensor 14A, illustrated in FIG. 3, is one example of a minimally invasive pressure sensor that can be attached to the patient via, for example, a radial arterial catheter inserted into an arm of the patient. In other examples, hemodynamic sensor 14A can be attached to the patient via a femoral arterial catheter inserted into a leg of the patient.

As illustrated in FIG. 3, hemodynamic sensor 14A includes housing 44, fluid input port 46, catheter-side fluid port 48, and I/O cable 50. Fluid input port 46 is configured to be connected via tubing or other hydraulic connection to a fluid source, such as a saline bag or other fluid input source. Catheter-side fluid port 48 is configured to be connected via tubing or other hydraulic connection to a catheter (e.g., a radial arterial catheter or a femoral arterial catheter) that is inserted into an arm of the patient (i.e., a radial arterial catheter) or a leg of the patient (i.e., a femoral arterial catheter). I/O cable 50 is configured to connect to hemodynamic monitor 12 via, e.g., one or more of I/O connectors 42 (shown in FIG. 2). Housing 44 of hemodynamic sensor 14A encloses one or more pressure transducers, communication circuitry, processing circuitry, and corresponding electronic components to sense fluid pressure corresponding to arterial pressure of patient 16 that is transmitted to hemodynamic monitor 12 (shown in FIG. 2) via I/O cable 50.

In operation, a column of fluid (e.g., saline solution) is introduced from a fluid source (e.g., a saline bag) through hemodynamic sensor 14A via fluid input port 46 to catheter-side fluid port 48 toward the catheter inserted into patient 16. Arterial pressure is communicated through the fluid column to pressure sensors located within housing 44 which sense the pressure of the fluid column. Hemodynamic sensor 14A translates the sensed pressure of the fluid column to an electrical signal via the pressure transducers and outputs the corresponding electrical signal to hemodynamic monitor 12 (shown in FIG. 2) via I/O cable 50. Hemodynamic sensor 14A therefore transmits analog sensor data (or a digital representation of the analog sensor data) to hemodynamic monitor 12 (shown in FIG. 2) that is representative of substantially continuous beat-to-beat monitoring of the arterial pressure of patient 16.

FIG. 4 is a perspective view of hemodynamic sensor 14B for sensing hemodynamic data representative of arterial pressure of patient 16. Hemodynamic sensor 14B is another example of hemodynamic sensor 14 (shown in FIG. 1). Hemodynamic sensor 14B, illustrated in FIG. 4, is one example of a non-invasive hemodynamic sensor that can be attached to the patient via one or more finger cuffs to sense data representative of arterial pressure of patient 16.

As illustrated in FIG. 4, hemodynamic sensor 14B includes inflatable finger cuff 52 and heart reference sensor 53. Inflatable finger cuff 52 includes an inflatable blood pressure bladder configured to inflate and deflate as controlled by a pressure controller (not illustrated) that is pneumatically connected to inflatable finger cuff 52. Inflatable finger cuff 52 also includes an optical (e.g., infrared) transmitter and an optical receiver that are electrically connected to the pressure controller (not illustrated) to measure the changing volume of the arteries under the cuff in the finger.

In operation, the pressure controller continually adjusts pressure within finger cuff 52 to maintain a constant volume of the arteries under the cuff in the finger (i.e., the unloaded volume of the arteries) as measured via the optical transmitter and optical receiver of inflatable finger cuff 52. The pressure applied by the pressure controller to continuously maintain the unloaded volume is representative of the blood pressure in the finger and is communicated by the pressure controller to hemodynamic monitor 12 (shown in FIG. 2). Heart reference sensor 53 measures the hydrostatic height difference between the level at which the finger is kept and the reference level for the pressure measurement, which typically is heart level. Accordingly, hemodynamic sensor 14B transmits sensor data that is representative of substantially continuous beat-to-beat monitoring of the arterial pressure waveform of patient 16.

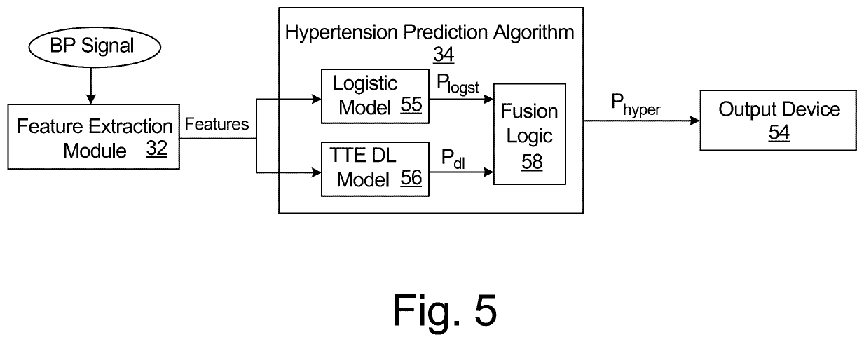

FIG. 5 is a schematic block diagram illustrating modules of hemodynamic monitoring system 10. FIG. 5 shows feature extraction module 32, hypertension prediction algorithm 34, and output device 54. Hypertension prediction algorithm 34 further includes logistic model 55, TTE DL model 56, and fusion logic 58.

Each of feature extraction module 32 and hypertension prediction algorithm 34 is a functional module of hypertension prediction software code 30, as shown in FIG. 1. Although hypertension prediction software code 30 is generally described herein as being divided into these two modules, in other examples, the functionality of hypertension prediction software code 30 could also be described as more or fewer modules, which could depend, in some examples, on how the code is written or organized. Additionally, any modules and/or sub-modules could also be entirely separate collections of code. The modules of hypertension prediction software code 30 will generally be described sequentially; however, these modules can also include overlapping or interspersed functionality.

Feature extraction module 32 is a first module of hypertension prediction software code 30 in hemodynamic monitoring system 10. Feature extraction module 32 includes methods in code for extracting features from a blood pressure waveform, such as an arterial pressure waveform. Feature extraction module 32 receives hemodynamic data in the form of a blood pressure signal (indicated as “BP signal” in FIG. 5) representative of a blood pressure waveform of patient 16 (shown in FIG. 1) as an input. The blood pressure signal corresponds to hemodynamic data sensed by one of hemodynamic sensors 14 and received by hemodynamic monitor 12. The sensed hemodynamic data to which the blood pressure waveform corresponds is passed to feature extraction module 32. Feature extraction module 32 extracts one or more features from the blood pressure waveform. In some examples, the sensed hemodynamic data may be smoothed and/or the extracted features may be validated. Feature extraction module 32 outputs one or more extracted features to hypertension prediction algorithm 34. In some examples, feature extraction module 32 outputs a subset of the extracted features to hypertension prediction algorithm 34.

Hypertension prediction algorithm 34 is a second module of hypertension prediction software code 30 in hemodynamic monitoring system 10. Hypertension prediction algorithm 34 determines a likelihood that patient 16 will experience an acute hypertensive event based on hemodynamic data from patient 16. Hypertension prediction algorithm 34 receives one or more extracted features from feature extraction module 32 as an input. As shown in FIG. 5, hypertension prediction algorithm 34 includes logistic model 55, TTE DL model 56, and fusion logic 58. Hypertension prediction algorithm 34 outputs an overall hypertension probability (“P_{hyper}”) to output device 54. P_{hyper} can be used to generate the hypertension index.

Hypertension prediction algorithm 34 includes several sub-modules, as illustrated in FIG. 5. Logistic model 55 is a first sub-module of hypertension prediction algorithm 34. Logistic model 55 uses one or more logistic regression models or functions to determine whether patient 16 is likely to experience an acute hypertensive event in the future. Logistic model 55 receives one or more extracted features from feature extraction module 32 as an input. Logistic model 55 passes a logistic model probability (“P_{logst}”) to fusion logic 58 of hypertension prediction algorithm 34. TTE DL model 56 is a second sub-module of hypertension prediction algorithm 34. TTE DL model 56 uses one or more deep learning models to determine whether patient 16 is likely to experience an acute hypertensive event within a certain time period. TTE DL model 56 receives one or more extracted features from feature extraction module 32 as an input. TTE DL model 56 passes a deep learning model probability (“P_{dl}”) to fusion logic 58 of hypertension prediction algorithm 34. Fusion logic 58 is a third sub-module of hypertension prediction algorithm 34. Fusion logic 58 produces a combined model of hypertension prediction algorithm 34. Fusion logic 58 combines the probabilities from logistic model 55 and TTE DL model 56 to determine an overall likelihood that patient 16 will experience an acute hypertensive event. That is, fusion logic 58 receives P_{logst} from logistic model 55 and P_{dl} from TTE DL model 56. Fusion logic 58 determines P_{hyper} from P_{logst} and P_{dl}. The value of P_{hyper} (or a variation or representation thereof) is the hypertension index.

Output device 54 is a device for receiving outputs from the modules of hypertension prediction software code 30. Output device 54 can include display 24, as shown in FIG. 1. For example, output device 54 can receive final probabilities for display via display 24. Output device 54 can receive outputs from hypertension prediction algorithm 34. More specifically, output device 54 can receive P_{hyper} (or the hypertension index). Outputs can be displayed via display 24, for example, as corresponding graphs representing the values over time.

Hemodynamic monitoring system 10 can include a pump that can provide intravenous or intra-arterial therapy to the patient. Hemodynamic monitor 12 can communicate with the pump (e.g. via a smart cable that is connected between hemodynamic monitor 12 and the pump). The pump can be an infusion device that can be connected to the patient through a tube cannulated within a vessel of the patient. The pump can be an infusion pump. For example, the pump can include a gravity infusion device, a syringe infusion pump, an elastomeric infusion pump, a volumetric infusion pump, a patient-controlled analgesia (PCA) pump, an ambulatory infusion pump, and/or any other kind of infusion pump.

Output device 54 can be connected or coupled to an infusion pump or similar device for administering fluid, medication, nutrients, etc. to patient 16. In some examples, output device 54 can be configured to automatically administer treatment to patient 16 via the infusion pump based on the value of P_{hyper}. For example, if P_{hyper} indicates a high likelihood patient 16 will experience an acute hypertensive event (e.g., based on predetermined criteria, as described above in reference to FIG. 1), output device 54 can automatically activate the infusion pump to administer a drug (such as a β-blocker, an α-adrenergic antagonist, a calcium channel blocker, an ACE inhibitor, a vasodilator, a diuretic, etc.) or another hypertension treatment to patient 16.

Healthcare worker 18 (shown in FIG. 1) can also interpret the value of P_{hyper} (or the hypertension index) from output device 54 to make a clinical decision for patient 16. In some examples, healthcare worker 18 can compare the value of P_{hyper} to predetermined criteria, as described above. Additionally or alternatively, healthcare worker 18 can respond to a sensory alarm that is invoked (e.g., sensory alarm 40 shown in FIG. 1).

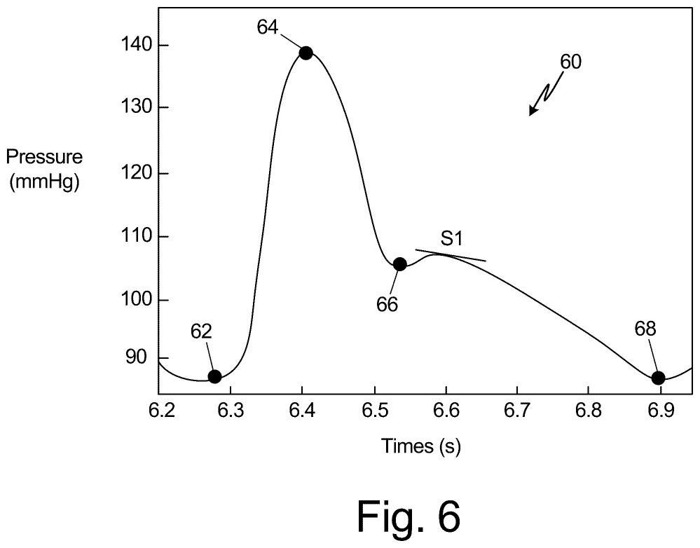

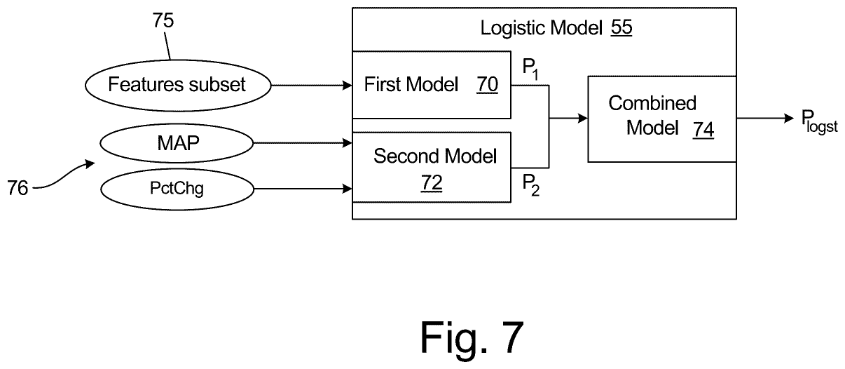

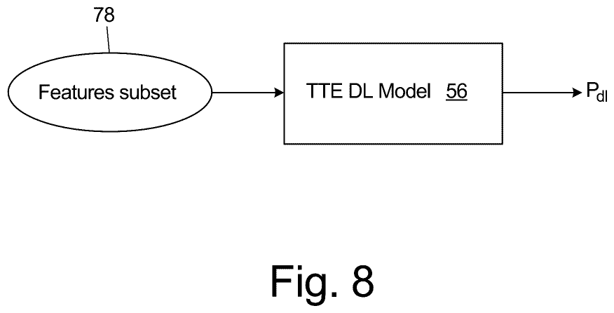

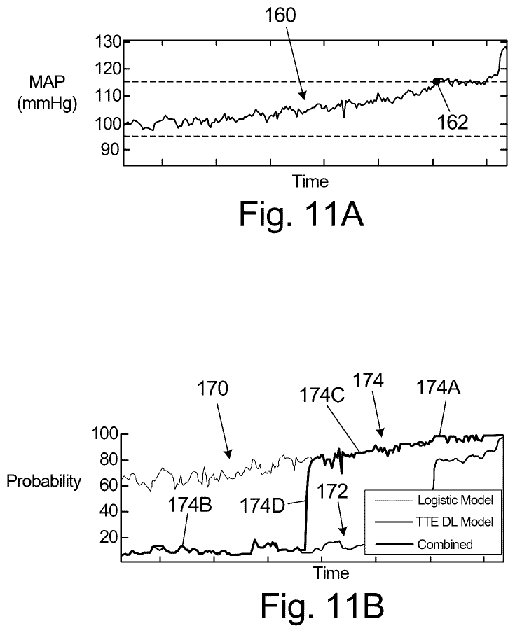

Modules of hypertension prediction software code 30 will be described in greater detail in turn below. Feature extraction module 32 will be described with reference to FIG. 6. Logistic model 55 will be described with reference to FIG. 7. TTE DL model 56 will be described with reference to FIGS. 8-10. Fusion logic 58 will be described with reference to FIGS. 11A-11B.

FIG. 6 is a graph illustrating an example trace of arterial pressure waveform 60 including example indicia 62-68 corresponding to a likelihood that a patient will experience an acute hypertensive event. Arterial pressure waveform 60 corresponds to hemodynamic data sensed by hemodynamic sensor 14 and received by hemodynamic monitor 12. Arterial pressure waveform 60 (e.g., represented via digital hemodynamic data) can include various features (or indicia) predictive of a future hypertensive event for patient 16. FIG. 6 shows indicia 62-68. Features are extracted from arterial pressure waveform 60 via feature extraction module 32, as discussed above with respect to FIG. 5. Prior to extracting indicia from arterial pressure waveform 60, beat detector algorithms identify the start and end of individual heartbeats for each waveform. Beat detection algorithms can identify the start of the heartbeat based on the maximum arterial pressure (e.g., pulmonary artery pressure, or PAP), the minimum arterial pressure, the maximum or minimum rate of change in arterial pressure, and/or the second derivative with respect to time in the arterial pressure. After heartbeat identification within arterial pressure waveform 60, various indicia can be extracted from the waveform on an on-going, beat-to-beat basis.

Indicium 62 of arterial pressure waveform 60 corresponds to the start of a heartbeat. Indicium 64 of arterial pressure waveform 60 corresponds to the maximum systolic pressure marking the end of systolic rise. Indicium 66 of arterial pressure waveform 60 corresponds to the presence and pressure of the dicrotic notch marking the end of systolic decay. Indicium 68 of arterial pressure waveform 60 corresponds to the diastole of the heartbeat. Also shown in FIG. 6 is example slope S1 of arterial pressure waveform 60, which may also provide indicia. Slope S1 is depicted at one location but is representative of multiple slopes that may be determined at multiple locations along arterial pressure waveform 60.

Additional indicia predictive of a future hypertensive event for patient 16 can be extracted from arterial pressure waveform 60 by hypertension prediction software code 30 based on behavior of arterial pressure waveform 60 during various intervals. For instance, the interval from the maximum systolic pressure at indicium 64 to the diastole at indicium 66, as well as the interval from the start of the heartbeat at indicium 62 to the diastole at indicium 66 can be extracted from arterial pressure waveform 60. Hypertension prediction software code 30 may identify additional indicia from arterial pressure waveform 60 during various intervals. For instance, systolic rise (indicia 62-64), systolic decay (indicia 64-66), systolic phase (indicia 62-66), diastolic phase (indicia 66-68), and the heartbeat interval (indicia 62-68) can be determined by hypertension prediction software code 30. Such indicia may include MAP (an average calculated arterial blood pressure for a single cardiac cycle) or a percent change in MAP. The area under the curve and/or standard deviations of arterial pressure waveform 60 determined for the above-referenced intervals can also serve as additional indicia predictive of a future hypertensive event for patient 16. Further indicia predictive of a future hypertensive event may include combinatorial features combining two of more individual indicia, or inter-relationship features describing relationships between short time series (e.g., over twenty seconds) of two or more individual indicia.

Any one or more of the above-described features extracted from arterial pressure waveform 60 can be used as inputs to hypertension prediction algorithm 34, such as in logistic model 55 and/or TTE DL model 56, as will be described in greater detail below.

FIG. 7 is a schematic block diagram illustrating logistic model 55 of hypertension prediction algorithm 34. As shown in FIG. 7, logistic model 55 includes first model 70, second model 72, and combined model 74. FIG. 7 shows features subset 75 and features subset 76, including MAP and percent change in MAP (indicated by “PctChg” in FIG. 7), which are inputs to logistic model 55. A first probability (“P₁”) is an output from first model 70, and a second probability (“P₂”) is an output from second model 72. P₁ and P₂ are inputs to combined model 74. FIG. 7 further shows P_{logst}, which is an output of logistic model 55.

Logistic model 55 determines a likelihood patient 16 (shown in FIG. 1) will experience an acute hypertensive event in the future. As shown in FIG. 7, logistic model 55 includes first model 70, second model 72, and combined model 74. First model 70 determines a probability of a future acute hypertensive event based on a threshold MAP. Second model 72 determines a probability of a future acute hypertensive event based on a percent change in MAP. Combined model 74 combines an output from first model 70 and second model 72. In some examples, logistic model 55 uses both first model 70 and second model 72. In other examples, cither first model 70 or second model 72 may not be used.

First model 70 includes a first logistic regression function. First model 70 receives features subset 75, which is a first subset of extracted features from feature extraction module 32. First model 70 determines P₁, which represents a probability of a future acute hypertensive event that is defined based on equaling or exceeding a threshold MAP value. For example, the threshold MAP can be 115 millimeters of mercury (mmHg). Other examples can use a different threshold MAP. P₁ can be determined according to the following general formula:

$${P}_{1} = \frac{100}{1 + {e}^{- [ {β}_{0} + {\sum }_{i = 1}^{14} {β}_{i} {x}_{i} ]}} \qquad ( Equation 2 )$$

Where,
e is Euler's number (a mathematical constant approximately equal to 2.71828);
β₀ is a bias term;
xᵢ (for i=1, 2, . . . , 14; total number of features in the model) is an extracted feature from feature extraction module 32; and
βᵢ is a corresponding feature weight (i.e., coefficient).
As illustrated in Equation 2, P₁ can be determined using fourteen features. In other examples, P₁ can be determined using one, a selected number, or all of the extracted features, and some examples can include the use of features other than those set out above. That is, features subset 75 can include any one or more extracted features from feature extraction module 32. In some examples, features subset 75 includes one or more different features than features subset 76 (used for second model 72, as described below). In some examples, features subset 75 does not include the features of features subset 76.

In some examples, first model 70 can be a multi-threshold model, meaning that the threshold MAP can be selected from a range of MAP values. For example, the threshold MAP can be selected from 95-140 mmHg. Other examples can use a different range of MAP values. In this way, healthcare worker 18 (shown in FIG. 1) can select the threshold MAP for a particular implementation of hemodynamic monitoring system 10, such as for a particular patient 16. In some such examples, multiple threshold MAPs can be selected, and first model 70 can determine a different P₁ for each threshold. P₁ for a multi-threshold model (designated herein as “P₁”) can be determined according to the following modified formula:

$${P}_{1}^{′} = \frac{100}{1 + {e}^{- [ {β}_{0} + {\sum }_{i = 1}^{14} {β}_{i} {\hat{x}}_{i θ} ]}} \qquad ( Equation 3 )$$

Where,
e is Euler's number;
β₀ is a bias term;
\hat{x}_{iθ} (for i=1, 2, . . . , 14; total number of features in the model) is a transformed feature from feature extraction module 32; and
βᵢ is a corresponding feature weight (i.e., coefficient).

Assuming that each feature xᵢ (for i=1, 2, . . . , 14) of the general formula (Equation 2) is normally distributed with mean μᵢ and standard deviation σᵢ at the hypertension threshold of 115 mmHg, each feature xᵢ can be transformed by estimating the new mean and standard deviation (μ_{iθ}, σ_{iθ}) when the hypertension threshold is changed to θ (i.e., different than 115 mmHg). The transformed features x_{iθ} can then be substituted into the general formula (Equation 2) to calculate the modified probability at threshold θ. The feature transformation can be expressed as:

$${x}_{i θ} = ( \frac{{x}_{i} - {μ}_{i}}{{σ}_{i}} ) * {σ}_{i θ} + {μ}_{i θ} \qquad ( Equation 4 )$$

Equation 4 is equivalent to:

$${x}_{i θ} = ( \frac{{σ}_{i θ}}{{σ}_{i}} ) * {x}_{i} + ( {μ}_{i θ} - \frac{{σ}_{i θ}}{{σ}_{i}} * {μ}_{i} ) \qquad ( Equation 5 )$$

Given the features are “physiological,” the standard deviations are not expected to vary significantly at different thresholds, so it can be assumed that the ratio σ_{iθ}/σᵢ is close to 1, which means that the transformed features can be estimated as:

$${x}_{i θ} \approx {x}_{i} + π \qquad ( Equation 6 )$$

Where,
π=μ_{iθ}−μᵢ (π is not calculated explicitly, and x_{iθ} is estimated from the blood pressure signal by using a DC signal transform).
   The probability at threshold θ is calculated according to Equation 3 without changing the model parameters from Equation 2.

In operation, first model 70 of logistic model 55 consumes extracted features from feature extraction module 32 and produces a probability of a future acute hypertensive event for patient 16. The output of first model 70 is P₁ (either single threshold or multi-threshold, i.e., P₁′). P₁ is passed to combined model 74 of logistic model 55.

Second model 72 includes a second logistic regression function. Second model 72 receives features subset 76, which is a second subset of extracted features from feature extraction module 32. Second model 72 determines P₂, which represents a probability of a future acute hypertensive event that is defined based on equaling or exceeding a threshold percent change in MAP. For example, the threshold percent change in MAP can be 20%. Other examples can use a different threshold percent change in MAP. In some examples, the threshold percent change in MAP can be combined with other criteria, such as a minimum MAP value (e.g., MAP>95 mmHg and increasing by at least 20%). P₂ can be determined according to the following general formula:

$${P}_{2} = \frac{100}{1 + {e}^{- [ - 21.1 + 0.15 * MAP + 0.265 * pctChg ]}} \qquad ( Equation 7 )$$

Where,
e is Euler's number;
MAP is a mean arterial pressure extracted by feature extraction module 32 (MAP can be bounded to [70, 110] mmHg in Equation 7); and
PctChg is a percent change in MAP extracted by feature extraction module 32 (pctChg can be bounded to (−∞, 30] in Equation 7).
   As illustrated by Equation 7, P₂ is determined using two extracted features from feature extraction module 32: MAP and percent change in MAP. That is, features subset 76 includes MAP and percent change in MAP. In some examples, as described previously, features subset 76 can include different features than features subset 75.

In operation, second model 72 of logistic model 55 consumes extracted features from feature extraction model 32 and produces a probability of a future acute hypertensive event for patient 16. Specifically, second model 72 consumes MAP and a percent change in MAP. The output of second model 72 is P₂. P₂ is passed to combined model 74 of logistic model 55.

First model 70 and second model 72 of logistic model 55 can determine P₁ and P₂, respectively, based on features from the same portion (e.g., beat or beats) of a patient's arterial pressure waveform. That is, P₁ and P₂ may directly correspond. In some examples, P₁ and P₂ are determined concurrently (i.e., at the same or approximately the same time).

As illustrated in FIG. 7, combined model 74 combines P₁ and P₂ to determine P_{logst}. For example, combined model 74 can determine a maximum of P₁ and P₂. Accordingly, P_{logst} can be expressed as:

$${P}_{logst} = max ( {P}_{1} , {P}_{2} ) \qquad ( Equation 8 )$$

That is, P_{logst} will be equal to whichever probability is greater of P₂ and P₁. For example, P_{logst} may equal P₂ (i.e., P₂>P₁) if MAP is increasing by 20% or more but the MAP value is not greater than or equal to 115 mmHg.

P_{logst} is the overall output of logistic model 55. P_{logst} is passed to fusion logic 58 of hypertension prediction algorithm 34. P_{logst} can be output whenever hemodynamic data is received from patient 16 (shown in FIG. 1). In some examples, P_{logst} can be output continuously or nearly continuously.

Logistic model 55 predicts the likelihood of an acute hypertensive event based on a threshold MAP or percent change in MAP. For example, logistic model 55 can accurately predict the likelihood of an acute hypertensive event at high MAP values (e.g., ˜115 mmHg). Thus, hemodynamic monitoring system 10 and hemodynamic monitor 12, including hypertension prediction algorithm 34 utilizing logistic model 55, can improve patient care.

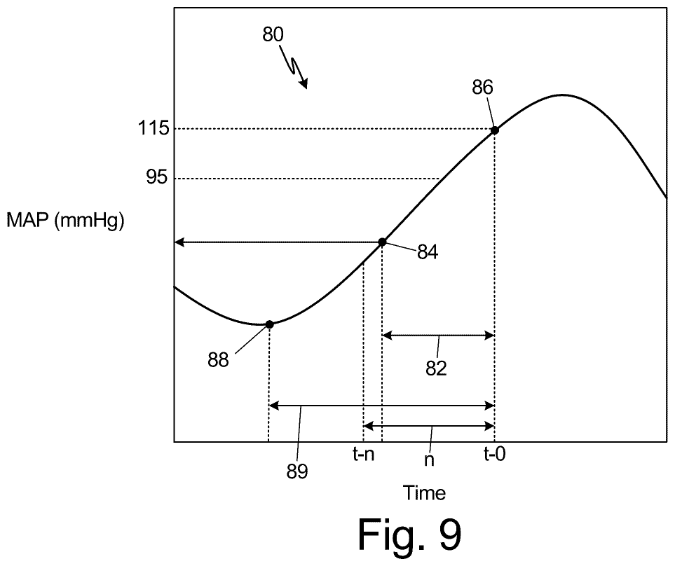

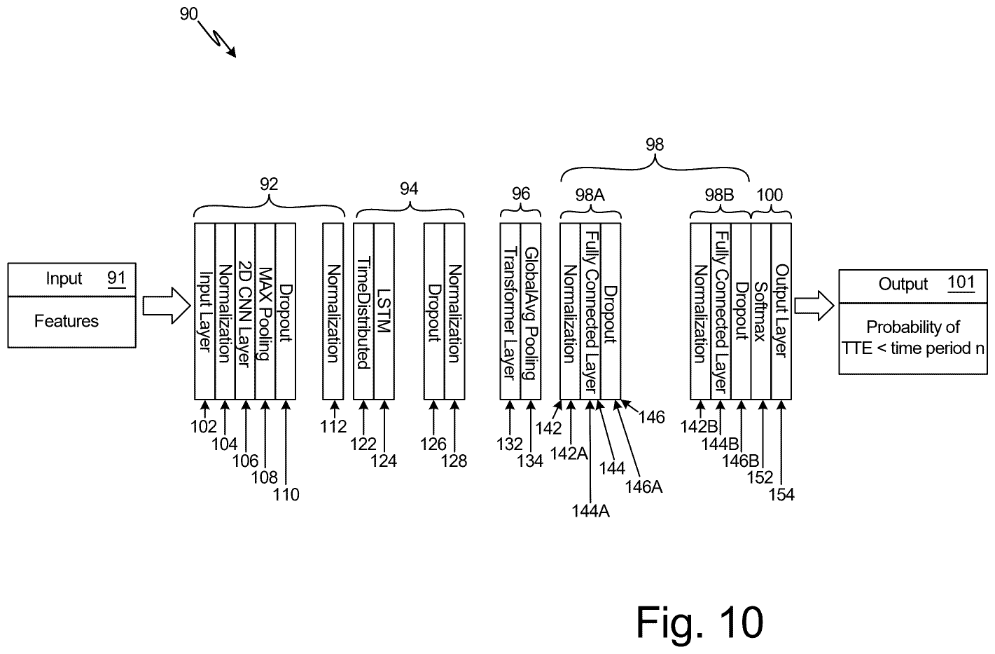

FIG. 8 is a schematic block diagram illustrating TTE DL model 56 of hypertension prediction algorithm 34. FIG. 9 is a graph illustrating MAP over time and showing a time-to-event (TTE). FIG. 10 is a schematic block diagram illustrating an example architecture of deep learning model 90 that may be used by TTE DL model 56. For clarity and case of discussion, FIGS. 8-10 will be described together. FIG. 8 shows TTE DL model 56 and features subset 78. FIG. 9 shows MAP trace 80, time period 82, sample point 84, hypertensive event point 86, sample point 88, time period 89, and time period n. FIG. 10 shows deep learning model 90, input 91, and output 101. Deep learning model 90 includes convolutional neural network (CNN) 92, long short-term memory network 94, transformer 96, one or more fully connected networks 98 (including fully connected network 98A and fully connected network 98B), and output network 100. Deep learning model 90 further includes layers 102, 104, 106, 108, 110, 112, 122, 124, 126, 128, 132, 134, 142 (including layers 142A and 142B), 144 (including layers 144A and 144B), 146 (including layers 146A and 146B), 152, and 154.

TTE DL model 56 includes one or more deep learning models. TTE DL model 56 determines a likelihood patient 16 (shown in FIG. 1) will experience an acute hypertensive event within time period n. In other words, TTE DL model 56 determines a probability of TTE<time period n. Time period n can be a period such as five minutes, six minutes, ten minutes, thirty minutes, an hour, or other periods. Time period n can be measured from the time hemodynamic data is received from patient 16. One example of time period n is shown in FIG. 9. It should be understood that the length of period n depicted in FIG. 9 is merely for illustrative purposes, and other time periods n are possible, as described previously.

For example, FIG. 9 shows MAP trace 80, which represents an example trace of MAP values measured from patient 16 over time. FIG. 9 shows hypertensive event point 86 indicating the onset of an acute hypertensive event (in this example, the point where MAP reaches 115 mmHg, although other thresholds could be used, as described previously) at time t−0. Time t−n is also indicated in FIG. 9. Accordingly, time period n extends from time t−n to time t−0.

Sample point 84 and sample point 88 indicate example time points when hemodynamic data might be measured from patient 16. Time period 82 represents the TTE from sample point 84 to hypertensive event point 86, and time period 89 represents the TTE from sample point 88 to hypertensive event point 86. The TTE is unknown when hemodynamic data is measured from patient 16, so the TTE is unknown at sample point 84 and sample point 88.

TTE DL model 56 is configured to determine the probability that time period 82 (and/or time period 89) is less than time period n. For example, if time period n is set at ten minutes, TTE DL model 56 would determine the probability that time period 82 (and/or time period 89) is less than ten minutes. As illustrated in FIG. 9, sample point 84 falls between time t−n and time t−0. In training TTE DL model 56, sample point 84 would be a positive sample because an acute hypertensive event would occur within a TTE that is less than time period n. In operation, TTE DL model 56 could determine a high probability that time period 82 is less than time period n. In contrast, sample point 88 does not fall between time t−n and time t−0. Thus, in training TTE DL model 56, sample point 88 would be a negative sample because an acute hypertensive event would not occur within a TTE that is less than time period n. Likewise, in operation, TTE DL model 56 could determine a low probability that time period 89 is less than time period n.

As shown in FIG. 8, TTE DL model 56 receives extracted features as an input and outputs P_{dl}. Specifically, TTE DL model 56 receives features subset 78, which is a third subset of extracted features from feature extraction module 32. The extracted features can be ten-minute (or other interval) samples, as measured back from the sample point (e.g., as indicated by the arrow extending back from sample point 84 in FIG. 9). For example, the extracted features can be averaged features over the ten-minute or other interval sample. In the example shown in FIG. 9, hemodynamic data (e.g., extracted features) corresponding to sample point 84 could include data corresponding to sample point 88, which is an inflection point where MAP begins to increase prior to hypertensive event point 86. In some examples, the ten-minute or other interval samples can also be overlapping (i.e., rolling) samples taken every few seconds, each minute, etc., to get continuous or nearly continuous samples. In one example, TTE DL model 56 is configured to use nine features. In other examples, TTE DL model 56 can be configured to use one, a selected number, or all of the extracted features, and some examples can include the use of features other than those set out above. That is, features subset 78 can include any one or more extracted features from feature extraction module 32. In some examples, features subset 78 includes one or more different features than features subset 75 (shown in FIG. 7) and/or features subset 76 (shown in FIG. 7). In some examples, features subset 78 does not include the features of features subset 75 or features subset 76.

Referring now to FIG. 10, FIG. 10 shows an example architecture of deep learning model 90 that may be used, for example, by TTE DL model 56. As shown in FIG. 10, deep learning model 90 consumes input 91. Input 91 can include the extracted features of features subset 78 (e.g., nine features from a ten-minute sample). Deep learning model 90 produces output 101. Output 101 can include P_{dl} representing the probability of TTE<time period n.

Deep learning model 90 can take the extracted features and pass them through one or more networks. Each network may include one or more constituent layers. For example, as shown in FIG. 10, deep learning model 90 can include CNN 92, long short-term memory network 94, transformer 96, one or more fully connected networks 98, and/or output network 100.

CNN 92 can include initial input layer 102, first normalization layer 104, two-dimensional (2D) CNN layer 106, maximum pooling layer 108, dropout layer 110, and/or second normalization layer 112. Input layer 102 can be the initial layer of the network where the raw input data (e.g., the extracted features of input 91) is fed. In some examples, the extracted features may be passed through input layer 102 as images and/or time-series data.

Input layer 102 can include a grid of neurons. Each neuron can correspond to a pixel in the input image or a feature of the time-series data. Each neuron of input layer 102 can be connected to a small local region of the input data. This local region can correspond to a small patch of pixels or data points in the image or time series. Each neuron can apply a convolution operation to its local region, combining the input values' corresponding weights to produce a single output value. Accordingly, input layer 102 can help CNN 92 capture local patterns and features in the input data (e.g., extracted features). In some examples, CNN 92 can apply an activation function, such as a Rectified Linear Unit (ReLU), to the output of each neuron from input layer 102 to introduce non-linearity into the network to enhance pattern recognition.

The local patterns may be normalized by first normalization layer 104. Normalization layer 104 can standardize the resulting outputs from input layer 102. CNN 92 may include one or more normalization techniques, such as batch normalization and/or some other technique. Batch normalization can normalize a subset of data and/or normalize activations across each feature map independently. Normalizing the data can stabilize training by reducing internal covariate shifts, allow for faster convergence, providing noise that can prevent overfitting, and avoid problems like exploding or vanishing gradients.

Two-dimensional CNN layer 106 may function similarly to input layer 102 in that it can convolve data sets by connecting neurons of data to different aspects of the data, which may be image data for two-dimensional CNN layer 106. Two-dimensional CNN layer 106 can generate one or more feature maps of the data, which can include corresponding two-dimensional representations of the data.

At maximum pooling (“MAX Pooling” in FIG. 10) layer 108, CNN 92 can reduce the spatial dimensions (e.g., width and/or height) of the input data. For example, maximum pooling layer 108 can extract a maximum value within a certain subregion of the data (e.g., a 2×2 window) and remove the other data. Accordingly, the system can reduce computational complexity and/or reduce overfitting. Additionally or alternatively, maximum pooling layer 108 can help extract dominant or clear features from the data. In some examples, an average pooling layer may be used additionally or alternatively to maximum pooling layer 108.

Dropout layer 110 can regularize the output data by randomly setting a fraction of the neurons in a layer to zero (e.g., during training). Accordingly, these neurons can be temporarily dropped out of the network. Dropping neurons can reduce the interdependence between or among neurons and avoid overreliance on specific features and, thus, make overfitting to the training data less likely. During training, the neurons may be dropped. Additionally or alternatively, during inference of test data, neurons may not necessarily be dropped but weights of neurons can be scaled a compensation factor to compensate for the fact that more neurons may be active during inference than during training. Dropping out neurons can introduce noise to the network during training and thus reduce fitting to noise present in the training data. This encourages the network to learn more robust features that are generalizable to unseen data.

Second normalization layer 112 may perform many of the features of first normalization layer 104. For example, normalization layer 112 can standardize the outputs from dropout layer 110. In some examples, the resulting normalized data can be used as inputs in long short-term memory network 94.

In some examples, deep learning model 90 can include a recurrent neural network (RNN). For example, deep learning model 90 can include one or more layers from long short-term memory network 94, each of which apply their layer operations independently to each time step of a sequence of the input data from the extracted features. For example, time distributed (“TimeDistributed” in FIG. 10) layer 122 can be part of long short-term memory network 94.

Long short-term memory network 94 can receive a sequence of data points with a temporal dimension, represented as a 3D tensor. These may be the output data, for example, from CNN 92. Time distributed layer 122 can wrap around each layer of long short-term memory network 94 (and/or of other layers in deep learning model 90) at each time step. Each wrapped layer (e.g., dense layer) can be applied independently. The same set of weights may be used for each time step. The output of time distributed layer 122 may be a sequence of outputs corresponding to the result of the wrapped layer of the corresponding time step of the input sequence.

Long short-term memory (“LSTM” in FIG. 10) layer 124 can capture long-range dependencies in the output sequential data, such as by maintaining a memory state across time steps. LSTM layer 124 can include memory cells that allow long short-term memory network 94 to remember information over long sequences. These memory cells may maintain a hidden state (e.g., a cell state), which may be updated and/or passed along to subsequent time steps. LSTM layer 124 can control the flow of information using one or more gates, such as a forget gate, an input gate, and/or an output gate. A forget gate can determine which information to discard from the cell state. An input gate can determine which new information to store in the cell state. Additionally or alternatively, an output gate can determine which information to output based on the current input and the cell state.

At each time step, LSTM layer 124 can generate update algorithms based on the input at the current time step, the previous hidden state, and/or the previous cell state. These update algorithms can determine how to update the cell state and the hidden state. LSTM layer 124 may apply one or more non-linear transformations to the input and the hidden state, such as the sigmoid and hyperbolic tangent (tanh) functions. These functions can help control the flow of information through the gates and to update the cell state.

During training, LSTM layer 124 can compute gradients and/or update parameters using backpropagation through time (BPTT). LSTM layer 124 can use BPTT over multiple time steps to learn the relationships between inputs at different time steps. In this way, LSTM layer 124 can capture long-range dependencies and remember information over extended sequences.

Dropout layer 126 may include one or more features of dropout layer 110. For example, dropout layer 126 may randomly set a portion of the neurons in a layer to zero during training. Additionally or alternatively, during inference dropout layer 126 can modify the weights of neurons. Normalization layer 128 can include one or more features of normalization layer 104 and/or normalization layer 112. For example, normalization layer 128 may standardize the outputs from dropout layer 126. Resulting normalized data can be used as inputs in transformer 96.

Transformer 96 can capture long-range dependencies in the received data from long short-term memory network 94. Unlike CNN 92 and long short-term memory network 94, which process their input data sequentially or hierarchically, transformer 96 includes a self-attention mechanism. Self-attention allows transformer 96 to weigh the importance of different words or tokens in a sequence of the received input data when processing each token. This mechanism allows transformer 96 to more fully capture contextual relationships among tokens, regardless of their position in the sequence.

Transformer layer 132 can include an encoder-decoder architecture. The encoder can process the input sequence. Additionally or alternatively, the decoder can generate an output sequence. In some examples, both the encoder and decoder include one or more layers of self-attention mechanisms and feed-forward neural networks.

Transformer layer 132 can, in some examples, include one or more multi-head self-attention layers. The multi-head self-attention layers can implement multi-head attention, which can include running multiple parallel self-attention operations. Each operation can focus on different aspects of the input sequence. Outputs from these parallel attention heads can be concatenated and/or linearly transformed to produce a final attention output. Positional encoding can be added to the input data to provide deep learning model 90 with information about the position of each token in the sequence.

In some examples, transformer layer 132 can include feed-forward neural networks (FFNs) in one or more of the multi-head self-attention layer(s). These FFNs can include one or more fully connected layers with a non-linear activation function, such as the ReLU function.

Global average (“GlobalAvg” in FIG. 10) pooling layer 134 can include one or more features of maximum pooling layer 108. In some examples, global average pooling layer 134 pools the layers using an average rather than pooling by a maximum value. For example, global average pooling layer 134 can extract an average value within a region of the data (e.g., a 2×2 window). In some examples, global average pooling layer 134 can remove the other data.

Fully connected networks 98 can be included as part of transformer 96 described above. In the example shown in FIG. 10, deep learning model 90 includes fully connected network 98A and fully connected network 98B, although other examples can include more or fewer fully connected networks 98. In some examples, fully connected networks 98 may include the FFNs described above. Additionally or alternatively, fully connected networks 98 can include additional features.

Fully connected networks 98 can include one or more normalization layers 142 to normalize the data, one or more fully connected layers 144, and/or one or more dropout layers 146. Specifically, fully connected network 98A includes normalization layer 142A, fully connected layer 144A, and dropout layer 146A. Fully connected network 98B includes normalization layer 142B, fully connected layer 144B, and dropout layer 146B. Normalization layers 142 may include one or more features of normalization layer 104 and/or normalization layer 128. Dropout layers 146 may include one or more features of dropout layer 110 and/or dropout layer 126.

Fully connected layers 144 can include one or more encoding filters that can perform mathematical operations to transform the input data into compressed data. Data samples of each element (e.g., extracted features or modified versions thereof) of the input data can be reduced by a target encoding factor by each of the encoding filters until fully compressed data reaches a latent space. Decoding filters can be applied to the compressed data in the latent space to yield output data. Data samples may be expanded by a target decoding factor (e.g., different from the target encoding factor) by each of the decoding filters until resulting in the output data. In some examples, an autoencoder model can autogenerate the architecture of the encoding filters and/or the decoding filters.

The fully connected deep learning autoencoder model may be part of an FFN described above. In fully connected networks 98, each node in one layer may be connected to every node in the subsequent layer. Additionally or alternatively, the output from each node can serve as an input to every node in the subsequent layer. Each node can represent a corresponding input value, such as an extracted feature or a modified feature thereof. Fully connected networks 98 can include one or more hidden layers between the input data and the latent space. Each layer can apply a weight or weighting to data for each node. This weight may be learned during a training phase of the model. Each hidden layer may apply one or more activation functions to the received interim data from the precedent layer. One or more of the activation functions may be a non-linear function, such as a trigonometric function. In some embodiments, the activation function includes a hyperbolic tangent (tanh) function.

During training, weights of each connection between layers (and/or of the activation functions and/or values thereof themselves) may be adjusted by backpropagating and/or gradient descent. Fully connected networks 98 can adjust these connection weights based on a goal of reducing or even minimizing a difference between a predicted output and an actual output.

Deep learning model 90 can include output network 100 in some examples. Output network 100 can include softmax layer 152 or some other non-linear function. Softmax layer 152 can include a multi-class activation function (vs. binary classification sigmoid function) to map any input value to a value between 0 and 1. Softmax layer 152 can introduce non-linearity into the output of deep learning model 90. Introducing non-linearity can improve the ability of deep learning model 90 to learn complex patterns and relationships in the data.

Softmax layer 152 can modify each element of output layer 154. Output 101 of output layer 154 can be interpreted as probabilities of generating specific values for each feature or pixel in the generated data. Output 101 can include P_{dl} described herein, or a slightly modified version thereof.

P_{dl} is the overall output of TTE DL model 56. P_{dl} is passed to fusion logic 58 of hypertension prediction algorithm 34. P_{dl} can be output whenever hemodynamic data is received from patient 16 (shown in FIG. 1). In some examples, P_{dl} can be output continuously or nearly continuously. Logistic model 55 (shown in FIG. 7) and TTE DL model 56 can determine P_{logst} and P_{dl}, respectively, based on features from the same portion (e.g., beat or beats) of a patient's arterial pressure waveform. That is, P_{logst} and P_{dl} may directly correspond. In some examples, P_{logst} and P_{dl} are determined concurrently (i.e., at the same or approximately the same time).

TTE DL model 56 predicts the likelihood of an acute hypertensive event occurring within time period n. For example, TTE DL model 56 can have accurate early prediction, e.g., when MAP is less than 95 mmHg and is trending upwards greater than 20%. TTE DL model 56 can also have accurate prediction in a MAP “grey zone” (e.g., in the range of about 95-115 mmHg) when MAP is relatively flat (e.g., trend <5%-10%) and there are no hypertensive events. Additionally, TTE DL model 56 can be very sensitive at lower MAP ranges, compared to logistic model 55, which may be more deterministic and less sensitive at lower MAP values. Thus, hemodynamic monitoring system 10 and hemodynamic monitor 12, including hypertension prediction algorithm 34 utilizing TTE DL model 56, can improve patient care.

FIG. 11A is a graph illustrating first trace 160 of MAP over time. FIG. 11B is a graph illustrating probabilities over time predicted by logistic model 55, TTE DL model 56, and a combined model produced by fusion logic 58, and corresponding to first trace 160. FIGS. 11A-11B will be discussed together. FIG. 11A shows first trace 160 and hypertensive event start 162. FIG. 11B shows logistic model probability 170, TTE DL model probability 172, and combined probability 174. Combined probability 174 includes regions 174A, 174B, 174C, and 174D.

Referring to FIG. 11A, first trace 160 can be an example of MAP measurements from patient 16 (shown in FIG. 1) over time. Referring to FIG. 11B, logistic model probability 170 can correspond to P_{logst} determined by logistic model 55. TTE DL model probability 172 can correspond to P_{dl} determined by TTE DL model 56. Combined probability 174 can correspond to P_{hyper} determined by a combined model that is produced by fusion logic 58. Each of P_{logst}, P_{dl}, and P_{hyper} can be equal to 100% when an acute hypertensive event is detected.

As shown in FIG. 11A, first trace 160 includes hypertensive event start 162 (based on a paradigm where hypertension is defined as MAP>115 mmHg). Hypertensive event start 162 corresponds to region 174A of combined probability 174 in FIG. 11B. The probability in region 174A is about 100% because an acute hypertensive event is detected. The probability leading up to region 174A is generally increasing.

In region 174B shown in FIG. 11B, combined probability 174 generally tracks TTE DL model probability 172. Region 174B corresponds to a portion of first trace 160 where MAP is relatively low. In region 174C shown in FIG. 11B, combined probability 174 generally tracks logistic model probability 170. Region 174C corresponds to a portion of first trace 160 where MAP is relatively high. Combined probability 174 illustrates a switch in the underlying model that is used (or prioritized) at region 174D shown in FIG. 11B. This switch can be based on the configuration of fusion logic 58, which receives P_{logst} from logistic model 55 and P_{dl} from TTE DL model 56.

Fusion logic 58 includes logic for combining the output of logistic model 55 (P_{logst}) and TTE DL model 56 (P_{dl}) into a combined model output (P_{hyper}), which can be represented as:

$${P}_{hyper} = f ( {P}_{logst} , {P}_{dl} ) \qquad ( Equation 9 )$$

Fusion logic 58 can include if/else logic or other logic for combining P_{logst} and P_{dl} to determine P_{hyper}. For example, the default output of the combined model can be an output from logistic model 55 (P_{logst}). In some examples, fusion logic 58 can be configured to switch the output of the combined model to an output from TTE DL model 56 (P_{dl}) when P_{dl} is relatively high (e.g., P_{dl}>30-50) and MAP is trending up. In some examples, fusion logic 58 can be configured to switch the output of the combined model to P_{dl} in a MAP “grey zone” (e.g., in the range of about 95-115 mmHg) when MAP is relatively flat (e.g., trend <5%-10%). Fusion logic 58 can also be configured to smooth the combined output (e.g., to avoid big jumps up or down). Fusion logic 58 can further include other logic or operations for combining P_{logst} and P_{dl} to determine P_{hyper}.

Fusion logic 58 combines the advantages of two machine learning models (logistic model 55 and TTE DL model 56) for predicting a likelihood of an acute hypertensive event for patient 16 (shown in FIG. 1) to bolster the overall predictive performance of hypertension prediction algorithm 34 (shown in FIGS. 1 and 5). Accordingly, fusion logic 58 allows hypertension prediction algorithm 34 to dynamically switch between models for improved predictions and to accommodate more prediction scenarios. Thus, fusion logic 58 can improve the usability of hemodynamic monitor 12.

FIG. 12 is a schematic block diagram illustrating training system 202 for training hypertension prediction algorithm 212. Training system 202 is situated within communication environment 200 including communication network 220, client system 230, system user 240, population of positive subjects 250, and population of negative subjects 254. Training system 202 includes hardware processor 204 and system memory 206 for storing hypertension prediction algorithm training software code 210. System memory 206 can include hypertension prediction algorithm 212 including predictive set of parameters 214, which can be arterial pressure waveform features or other features. Hypertension prediction algorithm 212 can be an example of hypertension prediction algorithm 34 described above with reference to FIGS. 1-11B. Network communication links 222 interactively connect client system 230 to training system 202 via communication network 220 and allow for the transmission of hemodynamic data 260 (which can include an arterial pressure waveform) from population of positive subjects 250 and population of negative subjects 254 to training system 202 via communication network 220.

System user 240 (who may be a medical professional, health care worker, or medical researcher, for example) may utilize client system 230 to interact with training system 202 over communication network 220. For example, system user 240 may receive hypertension prediction algorithm 212 (including predictive set of parameters 214) over communication network 220 and/or may download hypertension prediction algorithm training software code 210 to client system 230 via communication network 220. In one implementation, training system 202 may correspond to one or more web servers with accessibility over a packet network, such as the internet. Alternatively, training system 202 may correspond to one or more servers supporting a local area network (LAN) or included in another type of limited distribution network.

Hardware processor 204 is configured to execute hypertension prediction algorithm training software code 210 to receive hemodynamic data 260 (which can include an arterial pressure waveform) of each subject of population of positive subjects 250 and each subject of population of negative subjects 254. For example, positive subjects 250 can include one or more patients who experience an acute hypertensive event. In one example, acute hypertensive events can include time points where MAP has been greater than or equal to 115 mmHg for at least one minute. In another example, acute hypertensive events can include time points where MAP has increased by more than 20% and is greater than or equal to 90 mmHg for at least one minute. Negative subjects 254 can include one or more patients who do not experience an acute hypertensive event. In other words, negative subjects 254 include one or more patients who experience a non-event. For example, non-events can include time points that are relatively far away from any hypertensive events and where blood pressure is relatively flat (or steady). In one example, non-events can include time points at least twenty minutes away from any hypertensive events and where MAP is below 95 mmHg. In some examples (e.g., for training TTE DL model 56), positive subjects 250 can include one or more patients who experience an acute hypertensive event within time period n as described above (e.g., including all samples within time period n), and negative subjects 254 can include one or more patients who do not experience an acute hypertensive event within time period n.

Hardware processor 204 is further configured to execute hypertension prediction algorithm training software code 210 to define hemodynamic data 260 sets for use in training the hypertension prediction algorithm and extract waveform features from the arterial pressure waveform (of the hemodynamic data 260 sets) of the positive subject 250. In addition, hardware processor 204 is configured to execute hypertension prediction algorithm training software code 210 to transform the waveform features from positive subjects 250 to a plurality of parameters characterizing the waveform features. Hypertension prediction algorithm training software code 210 then identifies, from the plurality of parameters, predictive set of parameters 214 enabling prediction that the patient will experience an acute hypertensive event (e.g., identifies the waveform features that are most indicative in predicting an acute hypertensive event). The plurality of parameters characterizing the waveform features (extracted from the hemodynamic data) can be one, a combination of, or all of cardiac output, cardiac index, stroke volume, stroke volume index, pulse rate, systemic vascular resistance, systemic vascular resistance index, mean arterial pressure, baroreflex sensitivity measures, hemodynamic complexity measures, frequency domain hemodynamic features, and other possible features.

Identifying predictive set of parameters 214 from the plurality of parameters can include obtaining differential parameters (e.g., a second plurality of differential parameters) and/or generating combinatorial parameters (e.g., a third plurality of combinatorial parameters) and/or generating inter-relationship parameters (e.g., a fourth plurality of inter-relational parameters). The differential parameters are based on the plurality of parameters characterizing the waveform features and can be the same, partially the same, or different parameters than the plurality of parameters. The combinatorial parameters are generated using the plurality of parameters characterizing the waveform features and/or the differential parameters. In some examples, the combinatorial parameters can be a power combination of all or a subset of the plurality of parameters and the differential parameters, and the power combinations can include integer powers from among, for example, negative two, negative one, positive one, and/or positive two. The inter-relational parameters are generated over short periods of time using the plurality of parameters characterizing the waveform features and/or the differential parameters. Predictive set of parameters 214 can then be identified from the plurality of parameters, the differential parameters, the inter-relational parameters, and/or the combinatorial parameters to select a reduced set of parameters that are most indicative of predicting an acute hypertensive event. From the predictive set of parameters 214, hardware processor 204 can be configured to execute hypertension prediction algorithm training software code 210 to compute model coefficients corresponding to the predictive set of parameters to minimize the error of a hypertension index output by hypertension prediction algorithm 212, thereby further training hypertension prediction algorithm 212 to minimize error.

Hypertension prediction algorithm 212 (and hypertension prediction algorithm training software code 210), can include a machine learning model that that utilizes logistic regression (e.g., logistic model 55 shown in FIG. 7) and deep learning (e.g., TTE DL model 56 shown in FIGS. 8-10) techniques to identify predictive set of parameters 214 and determine the model coefficients, or another type of model for identifying predictive set of parameters 214 and determining the model coefficients that most accurately represent the likelihood that a patient will experience a future acute hypertensive event.

In some implementations, hardware processor 204 is configured to execute hypertension prediction algorithm training software code 210 to display hypertension prediction algorithm 212, the plurality of parameters characterizing hemodynamic data 260, and or predictive set of parameters 214 to system user 240 through display features available on client system 230. Additionally, hardware processor 204 is configured to execute hypertension prediction algorithm training software code 210 to update or otherwise modify predictive set of parameters 214 and/or model coefficients based on additional hemodynamic data 260 and/or other features received from one or more positive subjects of the population of positive subjects 250 and negative subjects of the population of negative subjects 254.

Trained hypertension prediction algorithm 212 can be validated on a held-out dataset to evaluate its predictive performance. For example, training system 202 can receive additional hemodynamic data from one or more negative subjects from the population of negative subjects 254 (subjects that did not experience an acute hypertensive event). Hardware processor 204 can then execute hypertension prediction algorithm training software code 210 to extract a predictive set of parameters (e.g., waveform features and/or other features) from the hemodynamic data. Hardware processor 204 can then execute hypertension prediction algorithm training software code 210 to determine the hypertension index utilizing the same model coefficients previously calculated and compare that hypertension index to a baseline hypertension index for a hypothetical negative subject who did not experience an acute hypertensive event. If the hypertension index is not within a margin of error of the baseline hypertension index, hardware processor 204 can then execute hypertension prediction algorithm training software code 210 to alter predictive set of parameters 214 and the model coefficients to more accurately predict the likelihood that an acute hypertensive event will occur, and training system 202 can then repeat the training steps with additional hemodynamic data from positive subjects 250 and/or negative subjects 254. This process can be repeated with different features and datasets until the prediction results from hypertension prediction algorithm 212 are satisfactory.

Although FIG. 12 shows hypertension prediction algorithm 212 as residing in or otherwise a part of system memory 206, other configurations can include a hypertension prediction algorithm that is copied to non-volatile storage (not shown in FIG. 12) or may be transmitted to client system 230 via communication network 220. Further, though client system 230 is shown as a personal computer in FIG. 12, such a configuration is provided merely as an example and other configurations may include client system 230 as or within a mobile communication device, such as a smartphone or tablet computer.

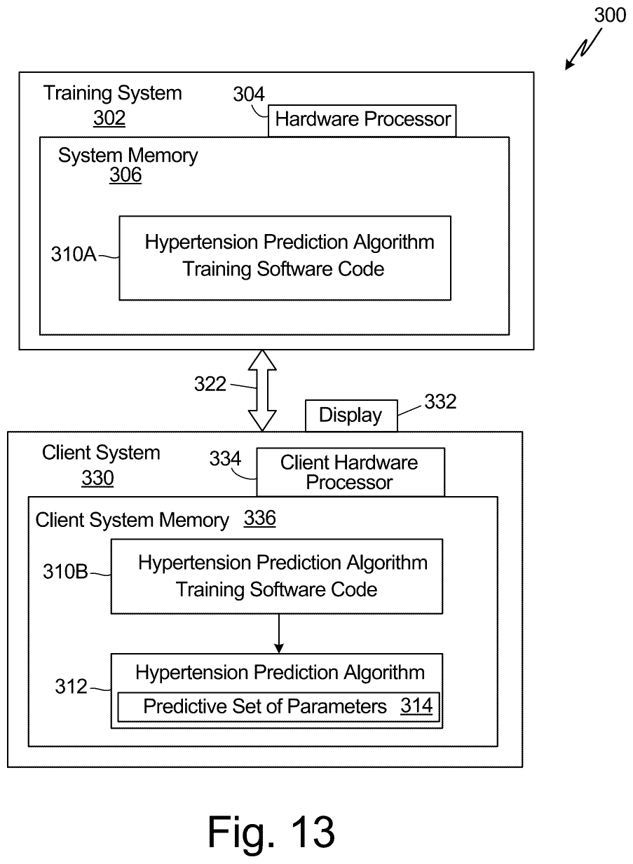

FIG. 13 is a schematic block diagram illustrating training system 302 for training hypertension prediction algorithm 312. FIG. 13 shows client system 330, which may be configured to train a hypertension prediction algorithm. Communication network 300 in FIG. 13 can include client system 330 interactively connected to training system 302 over network communication link 322. Training system 302 includes hardware processor 304 and system memory 306, which can be configured to store hypertension prediction algorithm training software code 310A. Client system 330 includes display 332, client hardware processor 334, and client system memory 336, which can be configured to store hypertension prediction algorithm training software code 310B. Hypertension prediction algorithm 312 includes predictive set of parameters 314.

Network communication link 322, training system 302, hardware processor 304, and system memory 306 correspond to network communication link 222, training system 202, hardware processor 204, and system memory 206 as shown in FIG. 12. In addition, hypertension prediction algorithm training software code 310A in FIG. 13 corresponds to hypertension prediction algorithm training software code 210 as shown in in FIG. 12 (i.e., hypertension prediction algorithm training software code 310A may share any of the characteristics attributed to hypertension prediction algorithm training software code 210).

Client system 330 corresponds to client system 230 as shown in FIG. 12, and hypertension prediction algorithm training software code 310B corresponds to hypertension prediction algorithm training software code 210 shown in FIG. 12 and/or hypertension prediction algorithm training software code 310A of training system 302 shown in FIG. 13. Thus, hypertension prediction algorithm training software code 310B and hypertension prediction algorithm 312 including predictive set of parameters 314 may share any of the characteristics attributed to hypertension prediction algorithm training software code 210 and hypertension prediction algorithm 212 including predictive set of parameters 214 as shown in FIG. 12.

Example training system 302 can have hypertension prediction algorithm training software code 310B located in client system memory 336, having been received from training system 302 via network communication link 322. One configuration includes network communication link 322 transferring hypertension prediction algorithm training software code 310B over a packet network. Once transferred (e.g., by being downloaded over network communication link 322), hypertension prediction algorithm training software code 310B may be persistently stored in client system memory 336 and may be executed locally on client system 330 by client hardware processor 334.

Client hardware processor 334 may be a central processing unit for client system 330, for example, so that client hardware processor 334 runs the operating system for client system 330 and executes hypertension prediction algorithm training software code 310B. In example training system 302, system user 240 (shown in FIG. 12) utilizing client system 330 can use hypertension prediction algorithm training software code 310B on client system 330 to identify predictive set of parameters 314 and determine model coefficients, thereby training hypertension prediction algorithm 312.

Additionally, system user 240 can utilize hypertension prediction algorithm training software code 310B on client system 330 to display hypertension prediction algorithm 312, parameters characterizing hemodynamic data 260 (shown in FIG. 12) and/or predictive set of parameters 314 on display 332. Display 332 can be a liquid crystal display (LCD), a light-emitting diode (LED) display, an organic light-emitting diode (OLED) display, or other display device suitable for providing information to system user 240 in graphical form.

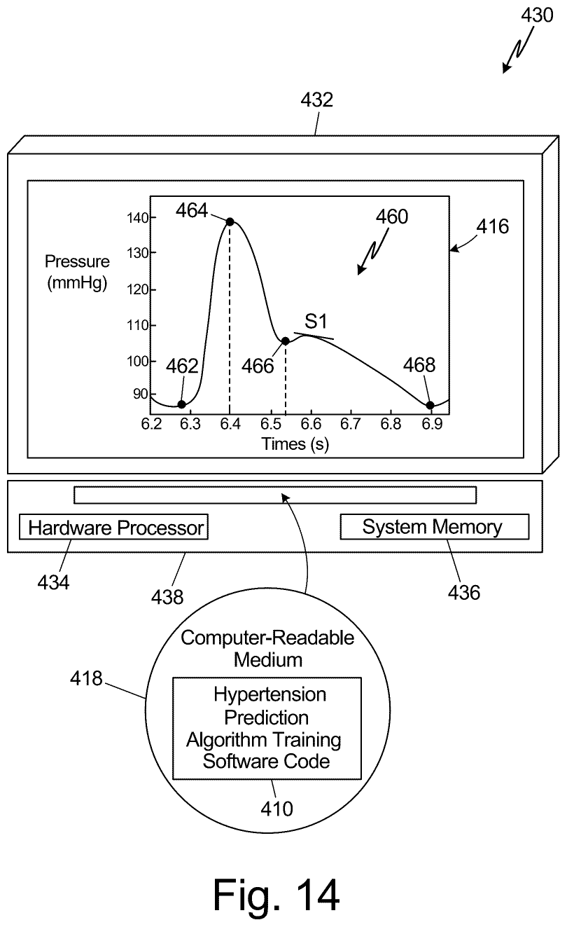

FIG. 14 is a schematic block diagram illustrating training system 430 and computer-readable medium 418 including instructions for performing model training. Training system 430 includes computer 438 having hardware processor 434 and system memory 436 interactively linked to display 432 (which can be similar to display 24, as shown in FIGS. 1-2, and display 332, as shown in FIG. 13). Training system 430 including hardware processor 434 and system memory 436 correspond to training system 202 and client system 230 of FIG. 12 and training system 302 and client system 330 of FIG. 13.

Parameters 416 characterizing hemodynamic waveform 460 (and potentially other features) are shown on display 432 and include features 462, 464, 466, 468, and S1 of hemodynamic waveform 460. Features 462, 464, 466, 468, and S1 correspond to features 62, 64, 66, 68, and S1 of FIG. 6 and are from a positive subject (i.e., a subject that experienced an acute hypertensive event). As described above with reference to FIGS. 6 and 12, in addition to features 462, 464, 466, 468, and S1 of hemodynamic waveform 460, additional indicia predictive of future hypertension in a patient can be extracted from hemodynamic waveform 460 based on behavior of hemodynamic waveform 460 in various intervals (set out with reference to FIG. 6 above). The features and intervals disclosed herein are provided only as examples and additional indicia predictive of future hypertension may be utilized.

Training system 430 includes computer-readable medium 418 with hypertension prediction algorithm training software code 410 stored thereon. In some examples, computer-readable medium 418 is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). Computer-readable medium 418 can include volatile and non-volatile computer-readable memories. Examples of volatile memories can include random access memories (RAM), dynamic random access memories (DRAM), static random access memories (SRAM), and other forms of volatile memories. Examples of non-volatile memories can include, for example, magnetic hard discs, optical discs, flash memories, or forms of electrically programmable memories (EPROM) or electrically erasable and programmable (EEPROM) memories.

As shown in FIG. 14, computer-readable medium 418 provides hypertension prediction algorithm training software code 410 for execution by hardware processor 434 of computer 438. Hypertension prediction algorithm training software code 410 corresponds to hypertension prediction algorithm training software code 210, 310A, and 310B and is capable of performing all of the operations attributed to hypertension prediction algorithm training software code 210, 310A, and 310B. For example, when executed by hardware processor 434, hypertension prediction algorithm training software code 410 is configured to identify predictive set of parameters 214, 314 and compute model coefficients, thereby training hypertension prediction algorithm 212, 312.

The systems and methods described herein can accurately predict the likelihood of an acute hypertensive event in a patient by using MAP with logistic regression and/or deep learning models. The models can be trained and implemented based on various MAP thresholds to accommodate patient-specific blood pressure management scenarios.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### DISCUSSION OF DETAILED EMBODIMENTS

The following are non-exclusive descriptions of possible embodiments of the present invention.

A system for predicting acute hypertension for a patient includes a hemodynamic sensor that produces, on an ongoing basis, a hemodynamic sensor signal representative of an arterial pressure waveform of the patient and an integrated hardware unit. The integrated hardware unit includes a system processor, a system memory, and a display including a user interface. The system memory includes instructions that, when executed by the system processor, cause the system to receive the hemodynamic sensor signal representative of the arterial pressure waveform of the patient; extract features from the arterial pressure waveform of the patient; determine, by a machine learning model, a probability of an acute hypertensive event of the patient based on the features extracted from the arterial pressure waveform; and output, to the display, an indication of the probability of the acute hypertensive event of the patient.

The system of the preceding paragraph can optionally include, additionally and/or alternatively, any one or more of the following features, configurations and/or additional components:

Wherein the acute hypertensive event is defined by one or more thresholds relating to a mean arterial pressure (MAP) of the patient.

Wherein a first threshold of the one or more thresholds is a MAP value of 115 millimeters of mercury (mmHg), and the acute hypertensive event is when the MAP of the patient equals or exceeds the first threshold.

Wherein a first threshold of the one or more thresholds is selectable from MAP values of 95-140 mmHg, and the acute hypertensive event is when the MAP of the patient equals or exceeds the first threshold.

Wherein a first threshold of the one or more thresholds is a MAP value that increases by twenty percent over a period of time, and the acute hypertensive event is when the MAP of the patient equals or exceeds the first threshold.

Wherein the period of time is twenty minutes.

Wherein the one or more thresholds include multiple thresholds, and the acute hypertensive event is when the MAP of the patient equals or exceeds any of the multiple thresholds.

Wherein the machine learning model includes a logistic model and a deep learning model.

Wherein the logistic model produces a probability that a MAP of the patient will equal or exceed a threshold MAP value.

Wherein the threshold MAP value is 115 mmHg.

Wherein the threshold MAP value is selectable from MAP values of 95-140 mmHg.

Wherein the logistic model produces a probability that a MAP of the patient will equal or exceed a threshold percent increase in MAP over a period of time.

Wherein the threshold percent increase in MAP is twenty percent.

Wherein the period of time is twenty minutes.

Wherein the logistic model includes a first model that produces a probability that a MAP of the patient will equal or exceed a threshold MAP value, and a second model that produces a probability that the MAP of the patient will equal or exceed a threshold percent increase in MAP over a period of time.

Wherein the threshold MAP value is 115 mmHg.

Wherein the threshold MAP value is selectable from MAP values of 95-140 mmHg.

Wherein the threshold percent increase in MAP is twenty percent and the period of time is twenty minutes.

Wherein the logistic model further includes a combined model that combines the probability produced by the first model and the probability produced by the second model.

Wherein the combined model determines a maximum of the probability produced by the first model and the probability produced by the second model.

Wherein one or more of the features extracted from the arterial pressure waveform are inputs for the logistic model.

Wherein the deep learning model produces a probability that a MAP of the patient will equal or exceed a threshold MAP value within a period of time.

Wherein the threshold MAP value is 115 mmHg.

Wherein the threshold MAP value is selectable from MAP values of 95-140 mmHg.

Wherein the period of time is 10 minutes.

Wherein one or more of the features extracted from the arterial pressure waveform are inputs for the deep learning model.

Wherein the machine learning model further includes fusion logic that combines a first output from the logistic model and a second output from the deep learning model into a combined output.

Wherein the combined output includes a default output based on the first output from the logistic model and switches to the second output from the deep learning model when a condition is satisfied.

Wherein the condition includes an upward trend in MAP of the patient, and the second output from the deep learning model is greater than thirty percent probability.

Wherein the condition includes an upward trend in MAP of the patient, and the second output from the deep learning model is greater than fifty percent probability.

Wherein the condition includes a MAP value of the patient between 95-115 mmHg, and an increase or decrease of ten percent or less in MAP of the patient.

Wherein the condition includes a MAP value of the patient between 95-115 mmHg, and an increase or decrease of five percent or less in MAP of the patient.

Wherein the combined output is a smoothed output.

Wherein the probability of the acute hypertensive event of the patient is represented by a hypertension index having a value between zero and one hundred.

Wherein the features extracted from the arterial pressure waveform are averaged features from a ten-minute sample of the arterial pressure waveform of the patient.

The system further includes an infusion pump, wherein the instructions further cause the system to activate the infusion pump to administer a hypertension treatment to the patient based on the probability of the acute hypertensive event of the patient.

A method for predicting acute hypertension for a patient in a system including an integrated hardware unit that includes a system processor, a system memory, and a display including a user interface includes receiving, on an ongoing basis from a hemodynamic sensor, a hemodynamic sensor signal representative of an arterial pressure waveform of the patient. The method further includes extracting features from the arterial pressure waveform of the patient and determining, by a machine learning model, a probability of an acute hypertensive event of the patient based on the features extracted from the arterial pressure waveform. The method further includes outputting, to the display, an indication of the probability of the acute hypertensive event of the patient.

The method of the preceding paragraph can optionally include, additionally and/or alternatively, any one or more of the following features, configurations and/or additional components:

Wherein the acute hypertensive event is defined by one or more thresholds relating to a mean arterial pressure (MAP) of the patient.

Wherein a first threshold of the one or more thresholds is a MAP value of 115 millimeters of mercury (mmHg), and the acute hypertensive event is when the MAP of the patient equals or exceeds the first threshold.

Wherein a first threshold of the one or more thresholds is selectable from MAP values of 95-140 mmHg, and the acute hypertensive event is when the MAP of the patient equals or exceeds the first threshold.

Wherein a first threshold of the one or more thresholds is a MAP value that increases by twenty percent over a period of time, and the acute hypertensive event is when the MAP of the patient equals or exceeds the first threshold.

Wherein the period of time is twenty minutes.

Wherein the one or more thresholds include multiple thresholds, and the acute hypertensive event is when the MAP of the patient equals or exceeds any of the multiple thresholds.

Wherein the machine learning model includes a logistic model and a deep learning model.

Wherein the logistic model produces a probability that a MAP of the patient will equal or exceed a threshold MAP value.

Wherein the threshold MAP value is 115 mmHg.

Wherein the threshold MAP value is selectable from MAP values of 95-140 mmHg.

Wherein the logistic model produces a probability that a MAP of the patient will equal or exceed a threshold percent increase in MAP over a period of time.

Wherein the threshold percent increase in MAP is twenty percent.

Wherein the period of time is twenty minutes.

Wherein the logistic model includes a first model that produces a probability that a MAP of the patient will equal or exceed a threshold MAP value, and a second model that produces a probability that the MAP of the patient will equal or exceed a threshold percent increase in MAP over a period of time.

Wherein the threshold MAP value is 115 mmHg.

Wherein the threshold MAP value is selectable from MAP values of 95-140 mmHg.

Wherein the threshold percent increase in MAP is twenty percent and the period of time is twenty minutes.

Wherein the logistic model further includes a combined model that combines the probability produced by the first model and the probability produced by the second model.

Wherein the combined model determines a maximum of the probability produced by the first model and the probability produced by the second model.

Wherein one or more of the features extracted from the arterial pressure waveform are inputs for the logistic model.

Wherein the deep learning model produces a probability that a MAP of the patient will equal or exceed a threshold MAP value within a period of time.

Wherein the threshold MAP value is 115 mmHg.

Wherein the threshold MAP value is selectable from MAP values of 95-140 mmHg.

Wherein the period of time is 10 minutes.

Wherein one or more of the features extracted from the arterial pressure waveform are inputs for the deep learning model.

Wherein the machine learning model further includes fusion logic that combines a first output from the logistic model and a second output from the deep learning model into a combined output.

Wherein the combined output includes a default output based on the first output from the logistic model and switches to the second output from the deep learning model when a condition is satisfied.

Wherein the condition includes an upward trend in MAP of the patient, and the second output from the deep learning model is greater than thirty percent probability.

Wherein the condition includes an upward trend in MAP of the patient, and the second output from the deep learning model is greater than fifty percent probability.

Wherein the condition includes a MAP value of the patient between 95-115 mmHg, and an increase or decrease of ten percent or less in MAP of the patient.

Wherein the condition includes a MAP value of the patient between 95-115 mmHg, and an increase or decrease of five percent or less in MAP of the patient.

Wherein the combined output is a smoothed output.

Wherein the probability of the acute hypertensive event of the patient is represented by a hypertension index having a value between zero and one hundred.

Wherein the features extracted from the arterial pressure waveform are averaged features from a ten-minute sample of the arterial pressure waveform of the patient.

The method further includes activating an infusion pump to administer a hypertension treatment to the patient based on the probability of the acute hypertensive event of the patient.

A method for use by a system for training a hypertension prediction algorithm to determine a hypertension index that represents a prediction that a patient will experience an acute hypertensive event. The system includes a hardware processor and hypertension prediction algorithm training software code stored in a system memory with the hardware processor configured to execute the hypertension prediction algorithm training software code. The method includes receiving hemodynamic data representing an arterial pressure waveform of a positive subject of a population of subjects including positive subjects that experienced an acute hypertensive event, and defining hemodynamic data sets for use in training the hypertension prediction algorithm with the hemodynamic data sets including the arterial pressure waveform collected from the positive subject. The hypertension prediction algorithm training software code executed by the hardware processor extracts waveform features from the arterial pressure waveform of the positive subject and transforms the waveform features from the positive subject to a first plurality of parameters characterizing the waveform features. A predictive set of parameters is identified from the first plurality of parameters, enabling prediction that the patient will experience the acute hypertensive event. The method further includes computing model coefficients to minimize an error of the hypertension index output by the hypertension prediction algorithm, thereby training the hypertension prediction algorithm.

The method of the preceding paragraph can optionally include, additionally and/or alternatively, any one or more of the following features, configurations and/or additional components:

Wherein the hypertension prediction algorithm includes a logistic model and a deep learning model.

Wherein the acute hypertensive event is defined by one or more thresholds relating to a mean arterial pressure (MAP) of the patient.

Wherein the hypertension prediction algorithm includes a logistic model.

Wherein the acute hypertensive event is defined by one or more thresholds relating to a mean arterial pressure (MAP) of the positive subject; a first threshold of the one or more thresholds is a MAP value of 115 millimeters of mercury (mmHg); and the acute hypertensive event is when the MAP of the positive subject equals or exceeds the first threshold.

Wherein the acute hypertensive event is defined by one or more thresholds relating to a mean arterial pressure (MAP) of the positive subject; a first threshold of the one or more thresholds is a MAP value that increases by twenty percent over a period of time; and the acute hypertensive event is when the MAP of the positive subject equals or exceeds the first threshold.

Wherein the hypertension prediction algorithm includes a deep learning model.

Wherein the acute hypertensive event is defined by one or more thresholds relating to a mean arterial pressure (MAP) of the positive subject; a first threshold of the one or more thresholds is a MAP value of 115 millimeters of mercury (mmHg); and the acute hypertensive event is when the MAP of the positive subject equals or exceeds the first threshold within a period of time.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A system for predicting acute hypertension for a patient, the system comprising:
a hemodynamic sensor that produces, on an ongoing basis, a hemodynamic sensor signal representative of an arterial pressure waveform of the patient; and
an integrated hardware unit comprising:
a system processor;
a system memory; and
a display including a user interface;
wherein the system memory includes instructions that, when executed by the system processor, cause the system to:
receive the hemodynamic sensor signal representative of the arterial pressure waveform of the patient;
extract features from the arterial pressure waveform of the patient;
determine, by a machine learning model, a probability of an acute hypertensive event of the patient based on the features extracted from the arterial pressure waveform; and
output, to the display, an indication of the probability of the acute hypertensive event of the patient.

2. The system of claim 1, wherein the acute hypertensive event is defined by one or more thresholds relating to a mean arterial pressure (MAP) of the patient, and wherein:
a first threshold of the one or more thresholds is a MAP value of 115 millimeters of mercury (mmHg), and the acute hypertensive event is when the MAP of the patient equals or exceeds the first threshold;
wherein a first threshold of the one or more thresholds is selectable from MAP values of 95-140 mmHg, and the acute hypertensive event is when the MAP of the patient equals or exceeds the first threshold; or
wherein a first threshold of the one or more thresholds is a MAP value that increases by twenty percent over a period of time, and the acute hypertensive event is when the MAP of the patient equals or exceeds the first threshold.

3. The system of claim 1, wherein the acute hypertensive event is defined by multiple thresholds relating to a mean arterial pressure (MAP) of the patient, and the acute hypertensive event is when the MAP of the patient equals or exceeds any of the multiple thresholds.

4. The system of claim 1, wherein the machine learning model includes a logistic model and a deep learning model.

5. The system of claim 4, wherein the logistic model produces a probability that a MAP of the patient will equal or exceed a threshold MAP value.

6. The system of claim 4, wherein the logistic model produces a probability that a MAP of the patient will equal or exceed a threshold percent increase in MAP over a period of time, wherein the threshold percent increase in MAP is twenty percent, and wherein the period of time is twenty minutes.

7. The system of claim 4, wherein the logistic model includes:
a first model that produces a probability that a MAP of the patient will equal or exceed a threshold MAP value; and
a second model that produces a probability that the MAP of the patient will equal or exceed a threshold percent increase in MAP over a period of time.

8. The system of claim 7, wherein the logistic model further includes a combined model that combines the probability produced by the first model and the probability produced by the second model, and wherein the combined model determines a maximum of the probability produced by the first model and the probability produced by the second model.

9. The system of claim 4, wherein one or more of the features extracted from the arterial pressure waveform are inputs for the logistic model and/or for the deep learning model.

10. The system of claim 4, wherein the deep learning model produces a probability that a MAP of the patient will equal or exceed a threshold MAP value within a period of time.

11. The system of claim 4, wherein the machine learning model further includes fusion logic that combines a first output from the logistic model and a second output from the deep learning model into a combined output, and wherein the combined output includes a default output based on the first output from the logistic model and switches to the second output from the deep learning model when a condition is satisfied.

12. The system of claim 1, wherein the probability of the acute hypertensive event of the patient is represented by a hypertension index having a value between zero and one hundred.

13. The system of claim 1, and further including an infusion pump; wherein the instructions further cause the system to activate the infusion pump to administer a hypertension treatment to the patient based on the probability of the acute hypertensive event of the patient.

14. A method for predicting acute hypertension for a patient in a system including an integrated hardware unit that includes a system processor, a system memory, and a display including a user interface, the method comprising:
receiving, on an ongoing basis from a hemodynamic sensor, a hemodynamic sensor signal representative of an arterial pressure waveform of the patient;
extracting features from the arterial pressure waveform of the patient;
determining, by a machine learning model, a probability of an acute hypertensive event of the patient based on the features extracted from the arterial pressure waveform; and
outputting, to the display, an indication of the probability of the acute hypertensive event of the patient.

15. The method of claim 14, wherein the acute hypertensive event is defined by one or more thresholds relating to a mean arterial pressure (MAP) of the patient, and wherein:
a first threshold of the one or more thresholds is a MAP value of 115 millimeters of mercury (mmHg), and the acute hypertensive event is when the MAP of the patient equals or exceeds the first threshold;
a first threshold of the one or more thresholds is selectable from MAP values of 95-140 mmHg, and the acute hypertensive event is when the MAP of the patient equals or exceeds the first threshold; or
a first threshold of the one or more thresholds is a MAP value that increases by twenty percent over a period of time, and the acute hypertensive event is when the MAP of the patient equals or exceeds the first threshold.

16. The method of claim 14, wherein the acute hypertensive event is defined by multiple thresholds relating to a mean arterial pressure (MAP) of the patient, and the acute hypertensive event is when the MAP of the patient equals or exceeds any of the multiple thresholds.

17. The method of claim 14, wherein the machine learning model includes a logistic model that produces a probability that a MAP of the patient will equal or exceed a threshold MAP value.

18. The method of claim 14, wherein the machine learning model includes a logistic model that includes:
a first model that produces a probability that a MAP of the patient will equal or exceed a threshold MAP value;
a second model that produces a probability that the MAP of the patient will equal or exceed a threshold percent increase in MAP over a period of time; and
a combined model that combines the probability produced by the first model and the probability produced by the second model, wherein the combined model determines a maximum of the probability produced by the first model and the probability produced by the second model.

19. The method of claim 14, wherein the machine learning model includes a logistic model and a deep learning model, and wherein one or more of the features extracted from the arterial pressure waveform are inputs for the logistic model and/or the deep learning model.

20. The method of claim 14, wherein the machine learning model includes a logistic model and a deep learning model, wherein the machine learning model further includes fusion logic that combines a first output from the logistic model and a second output from the deep learning model into a combined output, and wherein the combined output includes a default output based on the first output from the logistic model and switches to the second output from the deep learning model when a condition is satisfied.
